(19) United States
(12) Patent Application Publication
(10) Pub. No.: US 2016/0143879 A1
(43) Pub. Date: May 26, 2016

(54) METHODS OF TREATING ULCERS AND RELATED SYMPTOMS IN NON-HUMAN ANIMALS

(71) Applicant: Jaguar Animal Health, Inc., San Francisco, CA (US)

(72) Inventor: Serge Martinod, Groton, CT (US)

(21) Appl. No.: 14/931,320

(22) Filed: Nov. 3, 2015

Related U.S. Application Data
(60) Provisional application No. 62/074,956, filed on Nov. 4, 2014.

(51) Int. Cl. A61K 31/353; A61K 35/741; A61K 31/4439; A61K 45/06; A61K 36/47; A61K 9/50

(52) U.S. Cl. CPC A61K 31/353; A61K 36/47; A61K 9/5026; A61K 31/4439; A61K 45/06; A61K 9/5042; A61K 9/5047; A61K 35/741

(57) ABSTRACT

Provided are methods and compositions for treating and preventing ulcers, ulceratic conditions and/or the symptoms thereof, including gastric, intestinal, e.g., duodenal, and colonic ulcers in non-human animals, especially horses. The methods involve treating an afflicted animal a compositions comprising effective amounts of one or more acid reducing or inhibiting drugs, such as antacids and/or a proton-ion pump inhibitors, and a therapeutically effective amount of a proanthocyanidin polymer from Croton lechleri, or a Croton lechleri extract, in either enteric or non-enteric form. The ulcer treatment compositions are formulated as a paste, gel, or gel paste containing associated microparticles comprising antacid, proton-ion inhibitor, and C. lechleri proanthocyanidin polymer or C. lechleri extract, as well as other components, to particularly provide a colon targeted delivery system to treat and prevent ulcers in animals in need.

### RELATED APPLICATIONS

This application claims benefit of provisional patent application No. 62/074,965, filed on Nov. 4, 2014, the entire contents of which are incorporated herewith in their entirety.

### FIELD OF THE INVENTION

The invention relates to the treatment of ulcers and related symptoms in non-human animals, particularly equine animals, involving the use of compositions containing a combination of antacids, acid inhibitors and enteric or non-enteric forms of a proanthocyandin polymeric composition isolated from the plant Croton spp. or Calophyllum spp., or with an extract or latex derived therefrom. The compositions are useful in treating gastrointestinal and colonic ulcers in afflicted animals, thus providing a new, safe and effective treatment for ulcers, particularly ulcers of the colon, which are often difficult to detect and treat in large non-human animals, such as horses.

### BACKGROUND OF THE INVENTION

Ulcers, particularly gastrointestinal ulcers of the stomach, intestine and colon, and related conditions, can seriously affect the health and well-being of non-human animals, especially, large non-human animals, such as equine animals. Ulcers are lesions in the lining of the digestive tract and are very common in animals, particularly in horses that are used for activities involving endurance, competition and high-expectation performance.

Gastric ulcers, which occur in the stomach, and colonic ulcers, which occur in the hindgut, are common in large animals such as horses. Ulcers can also be present in the intestine, such as the duodenum, or proximal portion of the small intestine, of the animal. Gastric, intestinal and colonic ulcers (also known as hindgut ulcers) in equines such as horses can be a source of great pain and distress for the afflicted animal. The symptoms of ulcers can manifest themselves in young (e.g., foals) and older animals in various ways, depending upon the animal and its experiences. For example, athletic and performance horses (hunter/jumpers, dressage, endurance and western) routinely develop ulcers. However, studies have shown that even small changes in the routine of a non-performance (recreational) horse can cause ulcers in as few as five days. Thus, horses do not have to undergo intensive exercise, activity, or stress to develop ulcers. Only a few days of activities typically associated with recreational riding has been found to cause ulcers in horses tested for ulcers after their involvement in such activities.

Gastric ulcers can affect horses at any age. Foals are often susceptible because they secrete gastric acid as early as two days of age and the gastric fluid is highly acidic. Foals that have infrequent or interrupted feeding or that are recumbant for long periods of time, have been found to have gastric fluid of a lower pH, suggesting that milk has a protective effect against stomach acid and that recumbancy increases the exposure of the stomach to acid. (U.C. Davis Center for Equine Health Horse Report, www.thehorse.com).

Gastric ulcers are common in horses due to their anatomy and feeding conditions. The stomach of a horse is smaller than the stomachs of other species. Because of this, horses cannot handle large amounts of food; instead, their stomach better support grazing and ingestion of frequent, small portions of feed for extended periods of time. In a natural grazing situation, a horse requires a steady flow of acid for digestion; therefore, the stomach of a horse typically produces acid 24 hours a day, every day—up to 9 gallons of acidic fluid per day, even when the animal is not eating. In a natural, high-roughage diet, the acid produced is buffered by both feed and saliva. Id. However, when horses a fed on an “unnatural” schedule, such as only 2 times per day, for example, the stomach is subjected to a prolonged period in the absence of feed to neutralize the acid. In addition, high grain diets produce volatile fatty acids that also contribute to the development of ulcers.

Besides feeding and diet considerations, other risk factors for the development of gastric ulcers in non-human animals such as horses include physical and environmental factors, such as transport anxiety, stall confinement, intermittent feeding, lack of socialization. As determined by gastroscopy, a few hours of transport can induce gastric ulceration in horses than had no ulcers prior to departure. Moreover, chronic administration of non-steroidal anti-inflammatory drugs (NSAIDs) can decrease the production of a horse's protective gastric mucous layer, leading to ulcer susceptibility in the animal. Id.

The equine stomach is divided into the squamous region at the top, which is considered to be a continuation of the esophageal lining, and the glandular mucosal region at the bottom. It is the glandular bottom mucosal region that secretes gastric acid, as well as produces mucous and bicarbonate, which protect the mucosa from acid exposure. Consequently, although the bottom gastric region is exposed to acid for several hours per day, it is less common for ulcer formation, given the buffering action of the mucosa. Ulcers in the stomach bottom are often caused by chronic administration of NSAIDs. The top area of the stomach is involved with mixing the contents of the stomach and has less protection from produced acid. Thus, gastric ulcers are often found here. The stomach lining in the top portion is thin and has fewer mechanisms for acid protection. Because the horse's stomach produces acid at all times, even when the animal is not eating, the squamous mucosa is exposed to acid several hours a day, which can readily erode the lining of the top stomach region.

Definitive diagnosis of ulcers in an animal requires minimally invasive gastric endoscopy or gastroscopy, which allows evaluation of the gastrointestinal system, including the esophagus, the squamous and glandular regions of the stomach and the proximal section of the small intestine in horses. Feed material can impeded a complete endoscopic evaluation; therefore, an animal is usually fasted for a minimum of twelve hours prior to the procedure and examination.

While gastric ulcers and their treatment typically receive more attention, intestinal and colonic ulcers pose a significant problem for the digestive health of large animals and are frequently not properly or effectively diagnosed and/or are not readily treated. Often, animals, e.g., horses, that do not undergo intense exercise or stress develop colonic ulcers that remain undetected because a diagnosis of a colonic ulcer can be overlooked based on symptoms that can be similar to other types of non-ulcer-related conditions in the animal. In addition, detecting ulcers of the colon can be difficult in animals with large hind guts, particularly if the equipment or instruments necessary to perform scoping procedures is not readily available, if endoscopic procedures fail to be performed, and/or if the procedures themselves provide a negative or nonspecific result.

Because the economic and humane impacts of all types of ulcers, their symptoms and related conditions on afflicted animals are great, there is a compelling need for new, medically effective, as well as cost effective, treatments and remedies for gastric, intestinal and colonic ulcers, and their related conditions and associated symptoms in non-human animals such as horses. The present invention addresses such needs by providing advantageous methods and compositions to treat and prevent ulcers of the animal's digestive system.

### SUMMARY OF THE INVENTION

The present invention provides a method of treating ulcers and/or symptoms related thereto in a non-human animal, particularly, large animals such as horses, in which the method involves administering to an animal in need thereof a pharmaceutically acceptable composition comprising one or more agents that reduce or inhibit acid production in the gastrointestinal tract of the animal, in combination with an aqueous soluble proanthocyanidin from Croton lechleri, or an extract or latex derived from Croton lechleri, formulated in effective amounts to treat the ulcers and/or the symptoms thereof in the animal.

In an aspect, the composition further comprises one or more other drugs, compounds, or substances which may be incorporated into the composition, including, but not limited to, mucosal protectants, such as sucralfate; PGE₂ analogs, e.g., misoprostol; cytoprotective agents, such as bismuth subsalicylate, vitamins, e.g., Vitamins A, B1, B2, B6, B12, D3, E and K, particularly Vitamin E; natural products or herbal supplements, such as deglycyrrhizinated licorice; and/or a plant extract such as deglycyrrhizinated licorice, as described herein. In a preferred aspect, a mucosal protectant, e.g., sucralfate, is included in the compositions, in addition to the agents that reduce or inhibit acid production in the gastrointestinal tract of the animal and the aqueous soluble proanthocyanidin polymer from Croton lechleri or C. lechleri extract. In an aspect, the methods and compositions treat one or more of gastric ulcers, intestinal ulcers, such as duodenal ulcers, and colonic ulcers and/or the related symptoms in afflicted animals. In an aspect, the methods and compositions simultaneously treat gastric ulcers, intestinal ulcers, such as duodenal ulcers, and colonic ulcers and/or the related symptoms in afflicted animals.

In an aspect, the invention provides a method of preventing gastric, intestinal and colonic ulcers in non-human animals, particularly equine animals such as horses. The compositions of the invention can be administered to prevent first time ulcers or to prevent the recurrence of ulcers in the animals. The treatment and preventive methods of the invention may be used in conjunction with proper dietary and feeding oversight and management, as well as with methods of reducing and minimizing an animal's stress levels, for example, through better control and management of those aspects of an animal's environmental, lifestyle and living conditions that may lead to ulcerative conditions in the animal, for example, weaning, separation anxiety, housing, transportation, performance, endurance and competition.

The present invention preferably provides a method of treating and preventing ulcers of the digestive track in equine animals, such as horses. The animals may be young, such as foals, or they may be adult, since both young and adult horses develop ulcers from various causes. In an aspect, the method is used to simultaneously treat gastric, intestinal (duodenal) and colonic ulcers in horses diagnosed with ulcers. In an aspect, the method is suitable for treating and preventing colonic ulcers in a horse in need. In an aspect, the method is suitable for treating and preventing duodenal ulcers in a horse in need. In an aspect, the method is used to prevent, or prevent the recurrence of, gastric, intestinal, and colonic ulcers in a horse in need of treatment or prevention of an ulceratic condition. It will be understood that, in accordance with the invention, intestinal ulcers embrace ulcers of the small and large intestine, in particular, the duodenum, which is the proximal part of the animal's small intestine.

The methods of the invention also involve treating ulcers as well as diarrhea, which frequently accompanies an ulceratic condition in a non-human animal, with a combination of an aqueous soluble proanthocyanidin from Croton lechleri or a Croton lechleri extract, and a drug, substance, agent, or compound that acts to reduce, block, or inhibit the production of acid, particularly gastric acid, or acts to elevate the pH of the gastrointestinal environment. As used herein, the terms “drug,” “substance,” “agent,” and “compound” are considered interchangeable; the term “drug” is often used for convenience herein to indicate an active ingredient in the compositions and methods described. In various aspects, an anti-ulcer drug is an anti-acid (antacid) and/or a histamine type 2 (H-2) receptor antagonist such as cimetidine (TAGAMET), ranitidine (ZANTAC), nizatidine (AXID, AXID AR) and famotidine (PEPCID), which partially block acid production. In a preferred aspect, the drug is a proton pump (or proton-ion pump) blocking agent or inhibitor, such as omeprazole (PRILOSEC, GASTROGARD, ULCERGARD), rabeprazole (ACIPHEX, ACIFIX), esomeprazole (NEXIUM), or pantoprazole (PROTONIX), which arrest acid production. In an embodiment, the proton pump inhibitor is omeprazole (PRILOSEC, GASTROGARD, ULCERGARD). In other aspects, compositions comprising a combination of one or more antacids, H-2 receptor inhibitors and proton pump inhibitors are used in the methods to treat ulcers. In an aspect, a pharmaceutically acceptable composition comprising, in a gel or paste formulation, a combination of an effective amount of the aqueous soluble proanthocyanidin from Croton lechleri or Croton lechleri extract and an effective amount of one or more anti-ulcer drugs, such as antacids, H-2 receptor inhibitors and/or a proton pump inhibitors is provided to treat colonic, intestinal and gastric ulcers in an afflicted animal. In other aspects, the above compositions further comprise one or more other drugs, compounds, or substances which may be incorporated into composition, e.g., as components of the gel or paste formulation or as components of the nano- or microbeads/particles, including, but not limited to, mucosal protectants, such as sucralfate; a PGE₂ analog, e.g., misoprostol; cytoprotective agents, such as bismuth subsalicylate, vitamins, e.g., Vitamins A, B1, B2, B6, B12, D3, E and K, particularly Vitamin E; natural products or herbal supplements, such as deglycyrrhizinated licorice; and/or a plant extract such as deglycyrrhizinated licorice, as described herein.

In embodiments of the above methods, an extract from Croton lechleri is included in the compositions. In embodiments, the Croton lechleri extract in an enteric or non-enteric formulation. In other embodiments, a pharmaceutically acceptable C. lechleri proanthocyanidin polymer is included in the compositions. In an embodiment, the pharmaceutically acceptable C. lechleri proanthocyanidin polymer in the composition is an enteric formulation. In another embodiment, the pharmaceutically acceptable C. lechleri proanthocyanidin in the composition is a non-enteric formulation. In embodiments of the methods, the C. lechleri proanthocyanidin polymer is SB 300, SP 303, or crofelemer.

In embodiments, the non-human animal is a young or juvenile animal, or an adult animal. The method of the invention is not particularly limited as to the species of the animal and may include exotic, zoo, farm, or domestic animals, e.g., without limitation, equine, ovine, swine animals, goats, bison, buffalo, or camels, especially if such animal species are adversely affected by gastric, intestinal and/or colonic ulcers and related conditions. In particular or preferred embodiments, the non-human animal is an equine animal (a young or an adult horse).

In an embodiment, the C. lechleri proanthocyanidin polymer composition is a component of a composition or formulation comprising one or more antacid agents or proton pump inhibitors. In an embodiment, a C. lechleri extract is a component of a composition or formulation comprising one or more antacid agents or proton pump inhibitors. In embodiments, the composition or formulation contains other agents, such as mucoprotectants. In an embodiment, the non-human young or adult animal afflicted with ulcers is additionally suffering from diarrhea, e.g., diarrhea associated with an ulceratic condition. In other embodiments, the C. lechleri proanthocyanidin polymer or a composition comprising the C. lechleri proanthocyanidin polymer is provided in an amount of at least 50 mg to 250 mg.

As referred to herein, an antacid is an agent that neutralizes acidity, increases the pH, and/or reversibly reduces or blocks the secretion of acid by gastric cells to reduce acidity in the stomach or digestive tract. Antacids are commonly mixtures of magnesium, aluminum and calcium salts, as well as sodium in some cases, e.g., calcium carbonate, sodium bicarbonate, aluminum and magnesium hydroxide. Common antacids include, without limitation, ROLAIDS, TUMS, MAALOX, MYLANTA, GELUSIL, EQUATE, MILK OF MAGNESIA, PEPTO-BISMOL, and ALKA SELTZER.

As referred to herein, a histamine type 2 (H-2) receptor antagonist is an agent that partially blocks acid production. H-2 receptor antagonists competitively inhibit histamine binding at the H-2 receptors leading to a reduction in the secretion of gastric acid. Histamine stimulates the secretion of gastric acid by its action on H-2 receptors found on parietal cells of the gastric mucosa. Nonlimiting examples of H-2 receptor antagonists include cimetidine (TAGAMET), ranitidine (ZANTAC), nizatidine (AXID, AXID AR) and famotidine (PEPCID). H-2 receptor antagonists can be considered antacids that are suitable for use in the compositions and methods of the invention.

In a preferred aspect, the acid-reducing or inhibiting agent is a proton (proton-ion) pump inhibitor, which arrests acid production. Nonlimiting examples of proton pump inhibitors include omeprazole (PRILOSEC, GASTROGARD, ULCERGARD), rabeprazole (ACIPHEX, ACIFIX), esomeprazole (NEXIUM), or pantoprazole (PROTONIX), which are suitable for use in the compositions and methods of the invention. Without wishing to be limited by theory, proton pump inhibitor drugs stop production of stomach acid and provide longer lasting relief, but they do not neutralize gastric acid already present in the stomach. In other aspects, mucosal protectants, such as sucralfate; PGE₂ analogs, e.g., misoprostol; cytoprotective agents, such as bismuth subsalicylate, vitamins, e.g., Vitamins A, B1, B2, B6, B12, D3, E and K, particularly Vitamin E; natural products or herbal supplements, such as deglycyrrhizinated licorice; and/or a plant extract such as deglycyrrhizinated licorice, are also included.

In a particular embodiment, composition comprising the antacid and/or proton-ion pump inhibitor is in the form of a paste, gel, or gel paste suitable for oral administration. Preferably the paste or gel has at least two phases—liquid or oil based, in combination with a microsphere or micro/nano particle phase to deliver the anti-ulcer drugs throughout the digestive tract, including to the stomach, duodenum and the colon. Such pastes, gels, or gel pastes are typically and advantageously administered to the animal by topical application to the roof of the animal's mouth. In an embodiment, the C. lechleri extract or C. lechleri proanthocyanidin polymer composition is present in the gel, paste, or gel paste formulation, in combination with the one or more antacids and/or proton-ion pump inhibitors, for oral administration and delivery to the digestive system of an animal in need. In some cases, the gel, paste, or gel paste is contained in a delivery device, which can be a syringe, such as a needle-less syringe. In particular embodiments of the method as described herein, the gel, paste, or gel paste comprises polymeric microparticles or nanoparticles containing one or more antacids and/or proton-ion pump inhibitors and the C. lechleri extract, C. lechleri proanthocyanidin polymer, or composition thereof, as well as additional layers or coatings and components therein, to aid in delivering active agents to particular regions of an animal's gastrointestinal tract for ulcer treatment and prevention. In embodiments, the polymeric microparticles or nanoparticles are designed to provide controlled release of components and/or are pH-sensitive for effective delivery of active, ulcer-treating drugs to particular regions of an animal's digestive system.

In embodiments of the method, the C. lechleri extract or the C. lechleri proanthocyanidin polymer and the antacid and/or proton pump inhibitor drugs are co-formulated in the composition in amounts effective to reduce, decrease, diminish, inhibit, or block acid production and to thereby treat or prevent gastric, intestinal and/or colonic ulcers or ulcer production in the animal to which the composition is administered. In a preferred embodiment, the proton pump inhibitor is omeprazole (PRILOSEC, GASTROGARD, ULCERGARD).

In a particular aspect, the invention provides multicomponent composition for simultaneously treating or preventing gastric, intestinal and colonic ulcers in a non-human animal in need, which comprises (i) a paste or gel component comprising one or more drugs that reduce, block, or inhibit acid production and/or increase pH of the gastrointestinal tract in combination with an aqueous soluble proanthocyanidin polymer from Croton lechleri or a C. lechleri extract; and (ii) a microparticulate component admixed or associated with the paste or gel component, said microparticle component, which comprises (i) one or more drugs that block or inhibit acid production and/or increase pH, particularly in the colon; (ii) a layer or coating comprising one or more aqueous soluble, high molecular weight polymers; and (iii) a layer or coating comprising a pH sensitive substance or agent. In the microparticulate component, the high molecular weight polymer layer or coating encapsulates the one or more drugs that reduce or block acid production in the colon, thereby forming a core surrounded by an encapsulating layer; and the layer or coating comprising the pH sensitive substance or agent surrounds the encapsulating layer, thereby forming an enteric outer coating. The microparticulate component is in association with the paste or gel component so as to deliver the drugs to treat or prevent gastric, intestinal and colonic ulcers, especially colonic ulcers, in the animal.

In another particular aspect, acid reducing or blocking agents that are effective in the stomach, intestine and colon are provided in combination with the C. lechleri extract or C. lechleri proanthocyanidin polymer in a composition comprising multiple components, which are associated by their presence in a paste, gel, or gel paste to provide a colon-targeted drug delivery product. More specifically and as described herein, the product comprises a paste, a gel, or a gel paste composition which serves to associate at least two phases of the product. A first phase embraces a liquid or oil based paste or gel that contains antacids and/or acid blockers such as proton pump inhibitors (e.g., omeprazole, rabeprazole, esomeprazole, pantoprazole) and/or histamine H-2 receptor antagonists (e.g., ranitidine, famotidine, cimetidine and nizatidine) and/or cytoprotective and/or mucoprotective agents such as sucralfate, misoprostol and bismuth subsalicylate and/or natural products or herbal supplements, such as deglycyrrhizinated licorice and vitamins, such as Vitamins A, B1, B2, B6, B12, D3, E and K, particularly Vitamin E. A C. lechleri extract or C. lechleri proanthocyanidin polymer or composition is also preferably contained in the gel, paste, or gel paste. Associated with the gel or paste part of the colon-targeted delivery product is a second, multiparticulate system comprising micro- or nanoparticles and the like containing specific acid reducing and blocking drugs, e.g., antacids and proton pump inhibitors, and other active components, such as, for example, a C. lechleri proanthocyanidin polymer or a C. lechleri extract. The microparticulate system comprises at least two (protective) layers or coatings surrounding a core comprising the drugs and other active components to ensure active drug release and effectiveness in target regions of the gastrointestinal tract of the animal. One of the layers or coatings comprises one or more pH-sensitive polymers to prevent the burst release of drugs in the stomach or the proximal part of the small intestine. Another of the layers or coatings comprises high molecular weight, polymeric carbohydrates, e.g., polysaccharides, such as cellulose or ethylcellulose, to allow drug release in different parts of the cecum-colon, for example, after hydrolysis by bacterial enzymatic degradation.

In an embodiment, an extra layer of the microparticulate system comprising liposomes or mucoadhesive drug carriers (e.g., chitosan based or coated microparticles) may be included in the delivery product to increase local drug delivery to inflamed tissue, if the drug itself does not bind to the surface of the ulcer in the colon, (e.g., by attaching to exposed protein like sucralfate). In embodiments, the pH sensitive layer or coating may comprise a substance or agent selected from methacrylic acid polymers, e.g., EUDRAGIT®, cellulose acetate phtalates, pH controlled silicone microspheres, sodium alginate, or pH responsive gelatins. In embodiments, combinations of compounds and other polymers, e.g., a methacrylic acid polymer and poly (D,L-lactide-co-glycolide), are provided in the microparticulate layers or coating to avoid the premature release and loss of the drug in the duodenum or ileum before reaching the colon. In embodiments, other polymers of plant, animal, fungal, algal or bacterial origin; and compounds that protect the mucosa and its lining may be included in the compositions of the invention as described herein.

In an aspect, the invention provides a method of treating or preventing at least one, or all, of gastric, intestinal and colonic ulcers in a non-human animal that has, or that is at risk of developing, ulcers of the gastrointestinal tract, comprising administering to the animal in need thereof an effective amount of the above-described composition of the invention. In an embodiment, the composition is orally administered. In an embodiment, the composition is administered to a young or adult animal, in particular, a horse, that has one or more of a gastric, intestinal, or colonic ulcer, and/or symptoms thereof, or that is at risk of developing said one or more ulcers and/or symptoms thereof. In other embodiments, the compositions of the invention may be co-administered with other drugs or agents, for example, probiotics, which may serve to increase the efficiency of bacterial enzyme degradation. It will be appreciated that a probiotic containing bacteria which produce enzymes that act on particular polysaccharides contained in the layer or coating comprising the microparticle component of the composition are particularly suitable.

In embodiments, the compositions and formulations of the method are provided to an animal in need over a predetermined time period, i.e., for chronic administration. In other embodiments, the compositions and formulations of the method are provided to an animal in need over a discrete period of time, i.e., acute administration, until the ulcers or related symptoms have abated, improved and/or are eradicated.

### BRIEF DESCRIPTION OF THE DRAWINGS

FIG. 1 depicts a schematic representation of the anti-ulcer product provided by the invention and used in the described methods to simultaneously treat ulcers and related conditions in the stomach, intestine and colon of animals, particularly horses, in need.

### DETAILED DESCRIPTION OF THE INVENTION

Ulceratic lesions in the mucosal lining of the digestive tract are very common in equine animals, e.g., horses, used for recreation, commercial and agricultural purposes. In particular, many competitive activities, including racing, dressage, show jumping, endurance events and western performance, lead to ulcers and related symptoms in horses participating in these activities. It has been found that, depending on the intensity of training, the prevalence of gastric ulcers in horses can vary from 10 to 90%. Apart from exercise, many factors contribute to the development of ulcers, for example, transport to and from show grounds, stall confinement in unfamiliar surroundings, and/or irregular feeding schedules.

Equine animals such as horses may suffer from various types of ulcers, gastric ulcers in the stomach, intestinal ulcers, particularly in the duodenum, and colonic or hindgut ulcers (also called right dorsal colitis). About 87% of horses involved in competitive activities and about 37% of horses not involved in competition, i.e., leisure or recreational horses, have gastric ulcers. Colonic ulcers are less well understood but are as equally damaging to an animal as the more spotlighted gastric ulcers, which historically have received more extensive research and treatment development. About 63% of horses have colonic or hindgut ulcers (F. L. Pellegrini et al., 2005, J. Equine Veterinary Science, 25(3):133-117). In the early stages of a colonic ulcer condition, horses typically present with non-specific signs of colic episodes, anorexia and lethargy. As the condition worsens, clinical signs can be manifest and include fever and diarrhea. About 54% of horses have both gastric and colonic ulcers, and about 97% of horses typically have either a gastric ulcer or a colonic ulcer. While ulcers of the stomach and colon are lesions in the mucosal lining of the gastrointestinal tract, they have different causes and symptoms, and have typically required different treatments. The present invention provides compositions and methods that can conveniently, efficiently and economically treat either or both types of ulcers.

While the stomach represents less than 10% of the total volume of the digestive tract of a horse, the hindgut (consisting of the cecum and colon) is very large and is vital to the horse's digestive process and overall health. Of import, the hindgut harbors a vast bacterial environment that converts fiber to energy. Indeed, the horse is a “hindgut fermenter,” which means that it obtains most of its energy by fermenting forage in its colon. Because the hindgut plays such a critical role in a horse's digestion and health, this digestive organ likely stands as the source of so many of the problems, namely, colonic lesions, that affect the animal's performance, behavior and overall lifespan. Colonic ulcers are common in horses, and although they can go undetected, they are frequently identified in animals that already suffer from gastric ulcers.

Like gastric ulceration, ulcers in the hindgut occur when the protective mucosal lining is compromised. The development of colonic ulcers has been attributed primarily to right dorsal colitis caused by the use of NSAIDs, for example, the common anti-inflammatory phenylbutazone (“Bute”), as noted by those in the field (e.g., www.succeed-equine.com). Bute reduces pain and inflammation by blocking prostaglandins released from damaged tissue. An adverse effect is that Bute also blocks prostaglandin release in normal, healthy tissues, including those responsible for the mucus production and blood flow regulation in the gut. Inhibiting the production of prostaglandins in healthy tissue causes ulcers to develop in the horse's right dorsal colon.

Another source of colonic ulcers and other problems of the hindgut can be attributed to parasites. However, anthelmintics used to treat parasitic infections are highly effective against the types of parasites that affect the hindgut. Thus, parasitic causes of colonic ulceration have nearly all been eliminated. In rare cases in which parasites are involved, the lesions that are created may lead to ulceration.

Hindgut acidosis can be another source of colonic ulceration. When large amounts of simple carbohydrates (sugar and starches) remain undigested and reach the hindgut, the fermentation of fiber by endogenous bacteria is disrupted. This starch overload, which is common in view of today's feeding practices and processed feeds, stimulates the production of lactic acid-producing bacteria. As a result, the overall pH of the hindgut is lowered, causing a condition called hindgut acidosis. Hindgut acidosis results in the death of fiber-digesting bacteria, which release endotoxins, and can be the precursor to colonic ulcers. The lowering of the pH from hindgut acidosis is thought to reduce mucus production, which leaves the surface of the digestive tract unprotected. In addition, the death of the normal fauna allows pathogenic bacteria to proliferate. Such iron-dependent pathogens can attach to the micro blood vessels that become exposed in the intestinal lining, leading to ulcers, as well as to colic and laminitis. Because hindgut acidosis can be directly attributed to the way in which horses are commonly fed today, this condition, as well as the resulting colonic ulceration, are very real risks for many horses.

There is a need in the veterinary field for new and improved medications and treatments for ulcers, such as gastric, intestinal (duodenal) and colonic ulcers, and particularly colonic ulcers, in recreational and performance animals such as horses. At present, the only FDA approved drug for the treatment and prevention of gastric ulcers is the proton-ion pump inhibitor, omeprazole (GASTROGARD® and ULCERGARD® from Merial), which increases gastric pH in treated animals. Other, non-FDA approved substances include the mucosal protectant sucralfate (Carafate) and the prostaglandin E₂ (PGE₂) analog misoprostol (Cytotec). In suspension or tablet form, sucralfate contains an aluminum salt and works by forming a barrier or coating over the ulcerous lesion which protects the ulcer from the acid of the stomach, thereby allowing it to heal. Misoprostol also decreases the amount of gastric acid produced in the stomach. However, use of the drugs that are routinely administered for gastric ulcer treatment is typically not effective in the treatment of colonic or hindgut ulcers.

A number of drawbacks and challenges exist in the development of drugs and medications for colonic ulcers in large animals such as horses. At present, there are no FDA-approved drug treatments for colonic ulcers in horses. Medication given for treating gastric ulceration in horses (i.e., anti-acids, proton-ion pump blockers such as omeprazole and H-2 receptor antagonists such as ranitidine) are highly likely not to be effective in treating colonic ulcers because their principal mechanism of action is to decrease gastric acidity. Another significant challenge of drug development for colonic ulcers is product formulation to deliver drugs with biological activity in the colon. Anatomical, physiological and biochemical factors can affect the activity of a drug in the gastrointestinal tract with major variations among species (Kararli, 1995, Biopharmaceutics and Drug Disposition, 16:351-380). For example pH, bile, pancreatic secretions, mucus and fluid volume, transit time, and length of the small intestine can render a drug ineffective.

Simple co-administration of certain available drugs for treating colonic ulcers and gastric ulcers can be detrimental. For example, sucralfate binds to the surface of ulcers, which allows it to attach to exposed proteins, and coats the ulcer, thus protecting the ulcer surface so healing can occur. It dissociates in the acidic environment of the stomach to produce sucrose octasulfate and aluminum hydroxide and form a protective gel. However, if sucralfate drug is administered after another anti-ulcer drug, e.g., omeprazole, has increased the gastric acid pH, its activity and effectiveness will be reduced. If sucralfate is administered before the histamine H-2 receptor antagonist, adsorption to aluminum hydroxide occurs, and their absorption in the gastrointestinal/colonic environment is reduced. Other obstacles to delivering drugs to the colon are the absorption and degradation pathways in the upper gastrointestinal tract of large animals such as the horse. Thus, for effectiveness in the stomach, intestine and/or colon, a drug needs to be protected from the pH and enzymatic environment.

In addition, the digestive tracts of large animals such as horses are unique and provide challenges to administering drugs whose activities are required in certain areas and regions of the digestive tract for effective treatments. For example, the digestive physiology of the horse is characterized by rapid gastric transit, rapid but intense enzymatic activity along the small intestine, and a long and intense microbial fermentation in the large intestine. Another unique feature of a horse's gastrointestinal tract is the cecum-colon ecosystem (Santos et al., 2011, Animal, 5:1, pages 48-56). It is estimated that 30% to 80% of the cecum and colonial microbial population is strictly anaerobic. The bacteria in this environment can be classified into cellulolytic, proteolytic, lactate-using and glycolytic bacterial types. The proportion of cellulolytic bacteria is greater in the cecum than in the lower part of the hindgut. Some cellulolytic bacterial strains, e.g., Ruminococcus flavefaciens, R. albus and Fibrobacter succigenes, are specific to the horse. Starch-utilizing bacteria (lactobacilli and streptococci) and lactate-utilizing bacteria tend to be lower in the cecum than in other parts of the hindgut.

As noted above, the treatment and prevention of colonic ulcers in the veterinary field, especially in horses, is stymied by the difficulties in formulating products to deliver drugs and anti-ulcer compounds that have biological activity in the colon. Anatomical, physiological and biochemical factors of a non-human animal can affect the activity of a drug or medicament in the animal's gastrointestinal tract (T. T. Kararli, 1995, Biopharmaceutics and Drug Disposition, 16:351-380). For example, factors such as pH, bile, pancreatic secretions, mucus and fluid volume, transit time and length of the small intestine can render an anti-ulcer drug or drug treatment ineffective in a large non-human animal such as a horse. Indeed, before reaching the equine colon, the drug or medicament for ulcer treatment must travel through about 23 meters of small intestine having a pH of from 6.7 to 7.9 in 1 to 4 hours. The length and volume of the colon (i.e., about 3-4 meters and 130 L, respectively) and the cecum (i.e., about 1 meter and 33 L, respectively), as well as the large volume of fluid secreted and recovered by the large intestine (100 L/day), are unique physiological characteristics of horses. The pH in the equine colon (i.e., about pH 7.2 to 7.4) is also higher than in other species of animals, such as humans. These characteristics of the horse, which make ulcer treatment difficult in such animals, are presented in tabular form below. For certain parameters, a comparison between horse and human is provided.

| | Stomach | Small Intestine | Colon and Cecum |
|---|---|---|---|
| Length | | 22 meters | 8 meters |
| Relative | | 75% | Cecum: 4% |
| length | | | Large colon: 11% |
| (%) | | | Small colon: 10% |
| Volume | 15 liters | 30-40 liters | Cecum: 35 L |
| | | | Large colon: 80-90 L |
| | | | Small colon: 10 L |
| Relative | 8.3 (Horse) | 19.4 (Horse) | Cecum: 19.4 |
| capacity | versus | versus | Large colon: 47.2 |
| (%) | 17 (Human) | 67 (Human) | Small colon: 5.5 |
| | | | versus |
| | | | Human cecum and |
| | | | colon: 17 |
| pH | 3.3-5.4 (Horse) | 6.7-7.9 | Cecum: 7.0 |
| | versus | versus | Large colon: 7.2-7.4 |
| | 1.5-3.5 (Human) | 5-7 (Human) | |
| Transit | limited | 35 mn to | 6 to 10 hours |
| time | | 3 hours | |
| Role | Partial digestion/ | Enzymatic | Cecum: cellulose |
| | breakdown of | digestion | digestion by bacteria |
| | food into chyme | (duodenum) and | Large colon: absorp- |
| | | absorption | tion of carbohydrates. |
| | | (jejunum) | Small colon: Absorp- |
| | | | tion of water |

The present compositions and methods are particularly suitable for fulfilling the need of new and effective therapies and delivery approaches for treating gastric, intestinal and colonic ulcers in non-human animals, such as horses.

The symptoms of ulcers in affected animals can be subtle and may include irritability and changes in attitude and behavior (for the worse), poor appetite and decreased performance and energy. These symptoms may also be exhibited with other symptoms such as lethargy; musculo/skeletal discomfort and pain; decline or deterioration in body condition and/or appearance (dull hair coat); weight loss; alterations in eating or drinking patterns; resistance to grooming; reluctance or refusal to performing certain tasks; and behavior indicating discomfort, such as pawing or laying down excessively. Still other signs that may correlate with ulcers in the animal include sensitivity in the flank area and girthiness; cribbing (windsucking); wood-chewing; and weaving in the stall, an (atypical) unwillingness to work or cooperate, and resistance under saddle. Foals afflicted with an ulcer may also grind their teeth or lay on their backs. Once an ulcer is suspected or determined in a non-human animal such as horses, ponies, camels, donkeys, or mules, particularly, horses, the methods of the invention are useful in treating gastric and colonic ulcers and/or the symptoms of such ulcers in the animal. That the inventive methods and combinations described herein effectively treat all types of digestive tract ulcers, including colonic ulcers, in horses is new and surprising, particularly in view of the difficulty in accessing the cecum-colonic region of the horse and delivering an active, effective ulcer treatment to the colon.

In an aspect of the present invention, pharmaceutically acceptable compositions and formulations are provided which deliver acid reducing or blocking drugs and compounds, together with an extract from Croton lechleri or a proanthocyanidin polymer from Croton lechleri, e.g., SP-303 as described further herein, into the non-human animals, e.g., horses, for the treatment of colonic ulcers as well as associated diarrhea, if present, in animals in need thereof. In an aspect, the SP-303 compound, or other C. lechleri-derived proanthocyanidin polymer compositions, or C. lechleri extract, described herein, can be combined with, or used in combination with, one or more of an antacid or a proton-ion pump inhibitor, such as omeprazole, to provide an anti-ulcer product for treating ulcers. Such products would also and advantageously treat diarrheal conditions that frequently accompany ulcers and related conditions in afflicted animals, like horses. The inventive compositions and formulations would provide a significant and much needed breakthrough in managing gastrointestinal and colonic ulcers and related symptoms and adverse effects in non-human animals, particularly equine animals such as horses.

The invention provides methods directed to treating gastric and/or colonic ulcers and the uncomfortable and often painful discomfort that they cause in both young and adult horses and other non-human animals. The methods are effective in reducing and/or alleviating the ulcers and the symptoms that afflict such animals in need thereof. In particular, the methods are directed to the treatment of ulcers in horses, particularly ulcers of the colon that can lead to blood loss, irritability and poor absorption of nutrients in ulcerated young and adult animals. Treatment and prevention of ulceratic conditions in horses with the methods and compositions of the invention can positively impact and improve the performance of horses that are expected to perform at peak proficiency, including leisure and recreational horses and show horses, especially training and race horses.

The invention further provides formulations and compositions suitable for treating and preventing ulcers in young and adult animals. Unless otherwise noted herein, use of the term “animal” herein denotes non-human, warm-blooded mammals of a number of different species. Without wishing to be limiting, “young” or “juvenile” animals are generally about one year of age or under one to two years of age.

It is also to be understood that, even if not specifically stated herein, the methods and compositions of the invention are intended for the therapy and the prevention of ulcers, particularly colonic ulcers, that afflict both young and adult animals, such as horses, that are prone or susceptible to, or are at risk of, developing ulcers, depending on their living situations, activities, or experiences. Preventative or proactive administration of the compositions and formulations according to the methods of the invention can be provided to an animal prior to, or around the time of, stressful events, performances, transport, or relocation, for example. Animals (horses) having a history of gastric and/or colonic ulcers may benefit from proactive treatment with the compositions of the invention to decrease or abrogate the chances of developing ulcers or the recurrence of ulceration. During and following a course of treatment with the compositions and methods described herein, an animal being treated can be monitored for a change in its clinical behavior to determine that the gastric and colonic ulceratic conditions are improved, reduced, or eliminated. Preferably, the animal is examined via endoscopy or gastroscopy to confirm improvement and/or healing of ulceratic lesions, and prior to discontinuing therapy with the methods and compositions of the invention. Endoscopic examination involves short-term tranquilization of the animal to reduce stress to the animal from the procedure. Thereafter, an endoscope is inserted through the animal's nostril and guided down the esophagus into the stomach wherein the light and camera on the endoscope's terminus allow observation of the stomach lining A complete endoscopic evaluation can take about 10 to 20 minutes and is safe for the animal.

Often, equine animals such as horses may not suffer from gastric ulcers but may, however, present with intestinal (duodenal) and/or colonic ulcers, which may go unobserved or undetected.

The present invention embraces therapeutic and preventative compositions to treat and avert ulcers in regions of the digestive tract, particularly, gastric, duodenal and colonic ulcers, and related symptoms in animals, especially horses, experiencing digestive discomfort, as well as in animals at risk of or prone to digestive or gastrointestinal problems or ulcers. Signs that can indicate a digestive problem such as gastric or colonic ulcers in an animal such as a young or adult horse include the following: weight loss and/or general decline in body condition; resistance under saddle; irritability and other changes in attitude; lack of energy and stamina; loss of appetite; behavior indicating discomfort, such as pawing or laying down excessively; and low-grade anemia. Because of the large size and the position of the hindgut, some of the symptoms that are frequently attributed to gastric ulcers are more likely to be signs of colonic ulcers, namely, girthiness; sensitivity in the flank area; and difficulty bending, collecting and extending. The compositions and methods described herein may be administered to an animal or horse that exhibits any of the foregoing symptoms and/or if appropriate testing indicates that its manure pH is low, since the animal or horse is likely to be suffering from colonic ulcers.

The methods and compositions of the invention provide a solution to a significant need for the animal industry, particularly for performance animals used for sport and competitive activities, for treating and preventing gastric, intestinal and colonic ulcers, and especially colonic ulcers that often are not readily diagnosed or determined, and for which few treatment options are available for afflicted animals.

The methods, compositions and treatments of the invention are particularly suitable for treating animals of a young age, as well as adult animals, that suffer from, or are vulnerable to developing, ulcers of the digestive tract, such as gastric, duodenal, or colonic ulcers, or all of these types of ulceratic conditions. In an embodiment, the animals are young or juvenile non-adult animals that are born, bred, raised and/or maintained in a domesticated, performance, event and/or agricultural setting, e.g., animals raised and maintained for commodities such as labor, sport, performance, endurance, or other commercial or non-commercial capacity. In an embodiment, the animals are adult animals. Nonlimiting examples of animals affected by gastrointestinal ulcers and treatable by the methods and formulations of the invention particularly include young (foals) and adult equine animals (horses). Other animals that may suffer from ulcers and benefit from treatment and prevention by the methods and compositions described herein include young camels (calves) and adult camels. In addition, young cattle (calves), pigs (piglets), sheep (lambs), goats (kids), horses (foals) and adult animals, including, cattle, steer, bison, buffalo, goats, sheep and rams, may be treated according to the methods of the invention. In particular embodiments, both young and adult animals may be administered the compositions comprising acid reducing or blocking agents or drugs and proanthocyanidin polymer compositions of the invention to prevent and treat gastrointestinal and particularly colonic ulcers and related conditions in an animal or population of animals having or suspected of having ulcers, for example, based on diagnostic testing and/or observations of behavior and symptoms indicative of ulceratic conditions as described herein.

The present invention provides new and innovative approaches and products for the simultaneous treatment of gastrointestinal and colonic ulcers in adult or in young non-human animals with physiologically and pharmaceutically acceptable formulations and compositions comprising a therapeutically effective amount of one or more anti-ulcer drug and a C. lechleri proanthocyanidin polymer or composition thereof, or a C. lechleri extract. In addition, the approaches of the invention are particularly suitable and useful for the unique features of the equine gastrointestinal tract as discussed above.

In an embodiment, a product is provided to administer to non-human animals and deliver to regions or areas of the digestive tract of an animal anti-ulcer drugs in combination with a C. lechleri proanthocyanidin polymer or composition, or C. lechleri extract such that the drugs and C. lechleri proanthocyanidin polymer or C. lechleri extract will be active and effective for treating, preventing, or reducing the risk of ulcers, ulcer formation, and related symptoms and conditions in an animal in need. The preparation is particularly suited for treating horses. In an aspect, the preparation comprises a paste or a gel which serves as a matrix for associating the gel or paste containing anti-ulcer drugs and a C. lechleri proanthocyanidin polymer or composition or C. lechleri extract with nano- or microparticles or granules and the like containing encapsulated drugs for treating ulcers in the colon. Such particles or granules are surrounded by two or more layers or coatings providing for activity of the ulcer drugs in the colonic environment to which they are delivered by means of the compositions and methods described herein.

In an aspect, the drug is an anti-acid (antacid) or a histamine type 2 (H-2) receptor blocker, such as cimetidine, ranitidine, nizatidine and famotidine, which partially block acid production. In a preferred aspect, the drug is a proton pump inhibitor, such as omeprazole, rabeprazole, esomeprazole, or pantopraxole, which arrest acid production.

In general terms, “treating” an animal according to the present methods refers to achieving or obtaining a desired physiologic and/or pharmacologic effect, whether prophylactic, therapeutic, or both. As used herein “treating” or “treatment” can refer to ameliorating, preventing, inhibiting, reversing, attenuating, alleviating, abrogating, minimizing, suppressing, reducing, decreasing, diminishing, stabilizing, eradicating, curing, or eliminating the deleterious effects of a disease or condition, or the progression or worsening of the disease or condition. For example, successful treatment may involve alleviating one or more symptoms of a disease or condition, although not necessarily all of the symptoms, of the disease or condition, or attenuating the symptoms or progression of the disease or condition. Curing or eliminating the disease or condition from the animal is an optimal outcome of the practice of the methods of the invention.

According to the invention, treatment of an animal in need thereof typically involves the use or administration of effective amounts or therapeutically effective amounts of antacids, proton-pump inhibitors, and a proanthocyanidin polymer or a proanthocyanidin polymer composition, or an extract or sap, preferably from a Croton spp. particularly C. lechleri. The C. lechleri proanthocyanidin polymer or extract may be either enteric or non-enteric. Effective amount refers to the quantity (amount) of the drug and/or the composition containing the drug and the like, that induces a desired response in the animal subject upon administration or delivery to the animal. Optimally, an effective amount produces a therapeutic effect in the absence of, or with little or virtually no, adverse effects or cytotoxicity in the animal. Alternatively, any adverse effects associated with an effective amount are optimally outweighed by the therapeutic benefit achieved.

The treatment methods are directed to ameliorating, preventing, inhibiting, reversing, attenuating, alleviating, abrogating, minimizing, suppressing, reducing, decreasing, diminishing, stabilizing, eradicating, curing, or eliminating gastric, intestinal and/or colonic ulcers and/or their associated symptoms that adversely affect the health, lifestyle and performance of young and adult animals, such as horses. In an embodiment, the ulcer to be treated is a gastric ulcer such as a stomach ulcer. In an embodiment, the ulcer to be treated is an ulcer in the intestinal region such as the duodenum or cecum. In an embodiment, the ulcer to be treated is a colonic or hindgut ulcer. In an embodiment, gastric, intestinal and colonic or hindgut ulcers are simultaneously treated. In other embodiments, the methods and compositions of the invention also and advantageously improve and treat diarrhea, e.g., secretory/watery diarrhea, that may be associated with ulcers and ulceratic conditions in an afflicted animal.

In general, due to the anatomy of the gastrointestinal tract of adult animals such as horses, ulceratic conditions affecting the components of the digestive system, such as the small and large intestines and the cecum, typically also cause diarrhea. Young animals, e.g., foals, that are less than about three months of age do not have fully competent large intestines and ceca as do adult animals; therefore, young animals tend to be more prone to diarrhea caused by ulceratic conditions. In general terms, a foal is an equine, particularly a horse, that is one year old or younger in age. In accordance with the invention, the described compositions and methods can be administered to an animal suffering from ulcers and/or related symptoms and also experiencing associated diarrhea so as to treat both the ulcers and the associated diarrhea.

Proanthocyanidins and Tannins Obtained from Plant Extracts

Proanthocyanidins are types of condensed tannins, which are found in a large number of plants and are classified as hydrolyzable or condensed. Tannins and, in particular, proanthocyanidins are contained in many plants used in traditional medicine as treatment or prophylaxis for diarrhea (See, e.g., Yoshida et al., 1993, Phytochemistry, 32:1033; Yoshida et al., 1992, Chem. Pharm. Bull., 40:1997; Tamaka et al., 1992, Chem. Pharm. Bull., 40:2092).

Proanthocyanidins are comprised of at least two or more monomer units that may be of the same or different monomeric structure. The monomer units (generally termed “leucoanthocyanidins”) are generally monomeric flavonoids which include catechins, epicatechins, gallocatechins, galloepicatechins, flavanols, flavonols, flavan-3,4-diols, leucocyanidins and anthocyanidins. The polymer chains are thus based on different structural units, creating a wide variation of polymeric proanthocyanidins and a large number of possible isomers (Hemingway et al., 1982, J. C. S. Perkin, 1:1217). Larger polymers of the flavonoid 3-ol units are predominant in most plants and often have average molecular weights above 2,000 daltons (Da), containing 6 or more units (Newman et al., 1987, Mag. Res. Chem., 25:118).

Proanthocyanidin polymers and proanthocyanidin are found in a wide variety of plants, especially those having a woody habit of growth (e.g., Croton spp. and Calophyllum spp.). A number of different Croton tree species, including Croton sakutaris, Croton gossypifolius, Croton palanostima, Croton lechleri, Croton erythrochilus and Croton draconoides, which are endemic to South America, produce a red viscous latex sap called Sangre de Drago (also known as Sangre de Grado) or “Dragon's Blood”. The red viscous latex sap is known for its medicinal properties. For example, U.S. Pat. No. 5,211,944 describes the isolation of an aqueous soluble proanthocyanidin polymer composition from Croton spp. (See also, Ubillas et al., 1994, Phytomedicine, 1:77). The isolation of an aqueous soluble proanthocyanidin polymer composition from Calophyllum inophylum in U.S. Pat. No. 5,211,944.

In an embodiment, an extract from C. lechleri, such as the viscous sap which is extracted from the C. lechleri tree or the herbal medicine, Sangre de Drago, may be used in the compositions described herein. Sangre de Drago obtained from Croton trees is available throughout the Amazon, especially in the upper jungle of Peru and Ecuador, where such trees grow rapidly and can reach heights of 30-45 feet in three years. The sap (or extract) can be harvested, derived, or obtained from the Croton trees, in a manner akin to that of obtaining rubber or maple syrup. More ecologically-minded farming techniques are used to improve the cycles in which trees from which the sap is derived are grown, tapped and felled for sustainability. As used herein, a C. lechleri extract can also refer to C. lechleri sap (e.g., the latex sap or viscous sap) extracted from the C. lechleri tree.

In an embodiment, a proanthocyanidin polymer from C. lechleri, or a composition thereof, for use in the compositions and methods of the present invention is crofelemer. Crofelemer (CAS 148465-45-6) is an oligomeric proanthocyanidin of varying chain lengths derived from the Dragon's Blood of Croton lechleri, a tree of the family Euphorbiaceae, which is sustainably harvested under fair trade work practices in the Amazon. It has an average molecular weight of approximately 1900 Da to approximately 2700 Da. The monomers comprising crofelemer comprise catechin, epicatechin, gallocatechin, and epigallocatechin. The chain length of crofelemer ranges from about 3 to about 30 units with an average chain length of about 8 units. Crofelemer has the chemical formula: (C₁₅O_{6,7}H₁₂)ₙ and a molecular mass of 860-9100 g/mol. The antisecretory mechanism of action of crofelemer involves the targeting and inhibition of two, distinct intestinal chloride channels, namely, the cystic fibrosis transmembrane regulator conductance (CFTR) channel, which is a cAMP-stimulated Cl⁻ channel, and the calcium-activated chloride channel (CaCC), as reported, for example, by Tradtrantip, L. et al., 2010, “Crofelemer, an Antisecretory Antidiarrheal Proanthocyanidin Oligomer Extracted from Croton lechleri, Targets Two Distinct Intestinal Chloride Channels”, Mol. Pharmacol., 77(1):69-78). A general structure of crofelemer is shown below. In the structure, an H at the R position of the structure signifies procyanidin; an OH at the R position of the structure signifies prodelphinidin.

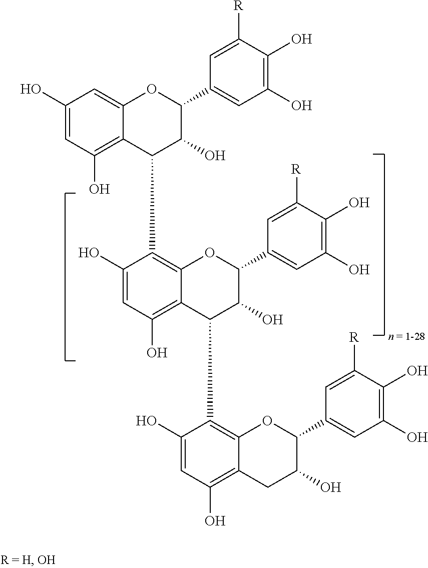

In accordance with an embodiment of the invention, crofelemer, or a pharmaceutically acceptable formulation or composition comprising crofelemer, is employed in the treatment methods and compositions as the proanthocyanidin polymer from Croton lechleri.

In an embodiment, SP 303, an oligomeric proanthocyanidin from Croton lechleri, (also known as crofelemer) is the proanthocyanidin polymer from Croton lechleri, or a pharmaceutically acceptable formulation or composition comprising SP 303, which is suitable for use in the treatment methods of the invention. SP-303 (R. Ubillas et al., 1994, Phytomedicine, 1:77-106) is largely composed of purified proanthocyanidin oligomers (−)-galloepicatechin and (+)-gallocatechin, (−)-epicatechin and (+)-catechin and is suitable for use in the enteric and non-enteric formulations and compositions for administration in the treatment methods described herein.

In another embodiment, SB 300, a proanthocyanidin polymer extract from Croton lechleri is the proanthocyanidin polymer from Croton lechleri, or a pharmaceutically acceptable formulation or composition comprising SB 300, which is suitable for use in the treatment methods of the invention. SB 300, as described, for example, by Fischer, H. et al., (2004, J. Ethnopharmacol., 93(2-3):351-357) provides a natural product extract that is particularly amenable for both enteric and non-enteric formulations and compositions, and is highly functional and cost-effective in the treatment methods described herein.

In an embodiment, a pharmaceutically acceptable composition comprising a proanthocyanidin polymer from Croton lechleri and employed in the treatment methods of the invention can be obtained from C. lechleri, e.g., as described in WO 00/47062 to Shaman Pharmaceuticals, Inc., the contents of which are incorporated herein, and formulated as a food or dietary supplement or nutraceutical formulation.

In other embodiments as noted above, compositions useful in the methods of the invention comprise a raw latex obtained from a Croton species or a Calophyllum species, or an extract or sap obtained from a Croton species or a Calophyllum species, which are not specifically polymeric proanthocyanidin polymer compositions. Exemplary extracts are described in Persinos et al., 1979, J. Pharma. Sci., 68:124 and Sethi, 1977, Canadian J. Pharm. Sci., 12:7.

In an embodiment, the proanthocyanidin polymer from Croton lechleri is formulated with an enteric coating or matrix in a variety of dosage formats known in the art (See, e.g., WO 00/47062 and U.S. Pat. Nos. 7,441,744 and 7,323,195, the contents of which are incorporated herein, and as briefly described below. In another embodiment, the proanthocyanidin polymer is formulation without an enteric coating or matrix. Both enteric and non-enteric forms of the proanthocyanidin polymer from Croton lechleri, for example, SB 300, are intended for use in the methods of the present invention.

### Preparation of Proanthocyanidin Polymer Compositions and Formulations

The proanthocyanidin polymer composition, effective for treating ulcers according to the invention, is comprised of monomeric units of leucoanthocyanidins. More particularly, the composition is comprised of proanthocyanidin polymers of 2 to 30 flavonoid units, preferably 2 to 15 flavonoid units, more preferably 2 to 11 flavonoid units and most preferably an average of 7 to 8 flavonoid units with a number average molecular weight of approximately 2500 Da. The proanthocyanidin polymer composition is preferably soluble in an aqueous solution. Preferred for use in the methods according to the invention is a proanthocyanidin polymer from C. lechleri; such a C. lechleri proanthocyanidin polymer may in some cases be in the form of a pharmaceutically acceptable composition.

Examples of proanthocyanidin polymeric compositions useful in the present invention are preferably isolated or purified from a Croton spp., namely, Croton lechleri, or Calophyllum spp. by any method known in the art. For example, the proanthocyanidin polymer composition may be isolated from a Croton spp. or Calophylum spp. by the method disclosed in U.S. Pat. No. 5,211,944 or in Ubillas et al. (1994, Phytomedicine, 1:77-106, called SP 303 therein), both of which are incorporated herein by reference. Other isolation methods are described in U.S. Pat. Nos. 7,556,831 and 8,067,041 (Example 2), the contents of which are incorporated herein. PCT application PCT/US00/02687, published as WO 00/47062, the contents of which are incorporated herein, also discloses a method of manufacturing a proanthocyanidin polymeric composition isolated from Croton spp. or Calophyllum spp. and enteric formulations of proanthocyanidin polymer dietary supplements, as well as methods of their preparation. Another illustrative method for isolating proanthocyanidin polymer from C. lechleri (such as crofelemer) is found in U.S. Pat. Nos. 7,341,744 and 7,323,195, the contents of which are expressly incorporated herein. As described above, the SP 303 and SB 300 purified forms of oligomeric proanthocyanidin polymer from Croton lechleri are suitable for use in the treatment methods of the invention.

In an embodiment, the proanthocyanidin polymer composition may be generally isolated by the following process, such as provided in U.S. Pat. No. 7,341,744, the contents of which are incorporated herein. Latex collected from Croton lechleri plants is mixed with purified water (preferably one part latex to two parts purified water). Any insoluble material in the latex solution is allowed to settle, e.g., by leaving the mixture at 4° C. overnight (12 hours). The supernatant is pumped away from the residue and is extracted with a short chain alcohol, such as n-butanol. The extraction is preferably performed multiple times, such as three times. After each extraction, the alcohol phase is discarded and the aqueous phase is retained. The aqueous phase is concentrated, for example, using an ultrafiltration device with a 1 kD cut-off membrane. This membrane can be a low protein binding cellulose membrane, or, alternatively, a polypropylene, teflon or nylon membrane can be used. The membrane used should be compatible with acetone. The purpose of the ultrafiltration is to remove the water from the material.

The retentate from the ultrafiltration is then concentrated to dryness, for example using tray-dryers at approximately 37° C. (±2° C.). The dried material is subsequently dissolved in water and is then chromatographed on a cation exchange column (e.g., a CM-Sepharose column) and a size exclusion column (e.g., an LH-20 column). In the preferred two column system, material is run over a CM-Sepharose and then an LH-20 column in a series. Specifically, the dissolved material is loaded onto the cation exchange column and is then washed with purified water. The proanthocyanidin polymer material is eluted from the cation exchange column with an aqueous acetone solution (preferably 30% acetone), thereby loading the proanthocyanidin polymer material onto the sizing column. The sizing column is disconnected from the cation exchange column and the material is then eluted off of the sizing column with an aqueous acetone solution (preferably 45% acetone). The fractions are collected and monitored with a UV detector, e.g., at a wavelength of 460 nm. Fractions containing the proanthocyanidin polymer material are combined and concentrated, for example, by ultrafiltration using, e.g., a 1 kD cut-off membrane (as described above for the ultrafiltration step prior to the chromatography steps). The retentate may then be concentrated to dryness using a suitable drying method, such as, but not limited to, a rotary evaporator, at a temperature of approximately 37° C. (±2° C.). Other suitable drying methodologies include, but are not limited to, tray drying and spray drying. Example 10 of U.S. Pat. No. 7,341,744 provides additional, non-limiting, methodology for preparing a composition comprising proanthocyanidin polymer, which can be used according to the invention. A detailed protocol for isolating an enriched proanthocyanidin polymer extract suitable for use in the methods of the invention is described in WO 00/47062 as noted herein above.

### Physiologically and Pharmaceutically Acceptable Compositions for Treating Ulcers of the Colon

In one embodiment, the anti-ulcer product for use in the methods for treating or preventing ulcers, particularly in the colon of the animal, is a paste or a gel composition, forming a matrix or milieu which serves to associate at least two phases of the composition. The first phase comprises a liquid or oil based paste or gel which contains one or more antacid and/or acid blockers such as proton-ion pump inhibitors. Non-limiting examples of proton pump inhibitors include, without limitation, omeprazole, rabeprazole, esomeprazole, or pantoprazole. The paste or gel may also contain histamine H-2 receptor blockers, examples of which include, without limitation, ranitidine, famotidine, cimetidine and nizatidine. Cytoprotective and/or mucoprotective agents, such as, for example, sucralfate, misoprostol and bismuth subsalicylate, and/or natural products, such as, without limitation, deglycyrrhizinated licorice, and vitamins, such as Vitamins A, B1, B2, B6, B12, D3, E and K, particularly Vitamin E, and/or extract of Croton lechleri, or the aqueous soluble proanthocyanidin polymer from Croton lechleri as described herein, are also intended to be included in the gel or paste phase. Other nonlimiting examples of mucoprotective agents include rebamipide (2-(4-chlorobenzoylamino-3-[2-(1H)-quinolinon-4-yl]proprionic acid), herbal demulcants and enprostil.

Admixed and/or otherwise associated or combined with the gel or paste phase is a multiparticulate preparation or composition, which constitutes a system especially for delivering colon-targeted anti-ulcer agents to the colon region of the animal's digestive tract. The multiparticulate preparation comprises nano- and/or microparticles, such as nanospheres, nanobeads, microspheres, and/or microbeads, that contain specific drugs. The particulate system comprises two protective layers or coatings: (i) a layer or coating comprising at least one pH-sensitive polymer, for example, without limitation, EUDRAGIT® (methacrylate polymers and methacrylic acid-methyl methacrylate copolymers), (Evonik Industries, Essen, Germany), which prevents the burst release of drugs, namely, the antacid and/or proton-ion pump inhibitor, in the stomach or the proximal part of the small intestine (the duodenum) and (ii) a layer or coating comprising high molecular weight polymers, such as complex carbohydrate polymers (i.e., glycans or polysaccharides), to allow drug release in different parts of the cecum-colon after hydrolysis by bacterial enzymatic degradation.

If the drug itself does not readily bind to the surface of the ulcer in the colon, for example, by attaching to an exposed protein like sucralfate, an additional phase or layer comprising liposomes or mucoadhesive drug carriers, such as chitosan-based or coated microparticles, may be included in the multiparticulate system to increase local drug delivery to inflamed tissues.

The pH sensitive polymer layer or coating may include, without limitation, pH sensitive polymers such as methacrylic acid polymers (EUDRAGIT®), or other poly(meth)acrylate polymers, cellulose acetate phtalates, pH controlled silicone microspheres, sodium alginate, or pH responsive gelatins. To avoid the premature release of the drug in the duodenum or ileum, which might result in drug release and loss before reaching the colon, combined polymers, for example, EUDRAGIT® and poly(D,L-lactide-co-glycolide), or newer-generation polymers, for example, EUDRAGIT® P4135-F that can dissolve at a pH greater than 7.2, may also be used. Such pH sensitive polymers are preferably alkali soluble and acid insoluble polymers of the types described.

Polysaccharides suitable for use in the formulations include, for example, cellulose for release in the cecum by cellulolytic bacteria and/or starch for release in other parts of the colon. Other polymers may be plant-derived, such as ethyl cellulose, pectin, inulin, locust bean gum, guar gum, amylose; or from algae, such as alginates; from fungi, such as sleroglucan; or from bacteria, such as dextran, cyclodextrin; or animal-derived, such as chondroitin sulfate, hyaluronic acid, or chitosan. Combinations of polysaccharides and/or polymers are encompassed by the formulations. To optimize the release profile and adapt to the specific conditions of the animal's (e.g., horse's) hind gut, a mixed coating or layer may be used, such as, for example, a coating or layer comprised of pectin-ethylcellulose, guar gum-ethylcellulose, amylose-ethylcellulose, pectin-chitosan, polysaccharide-EUDRAGIT®, or pectin-chitosan-hydroxypropylmethylcellulose (HPMC).

Because the composition of the colonic microflora can also be altered in disease conditions such as colitis and colonic ulcers, the composition of the coating will be specific to the disease condition. In addition, to increase the efficiency of the bacterial enzyme degradation, a probiotic which contains bacteria and/or yeast that produce one or more enzymes suitable or appropriate for a given polysaccharide of the coating may be co-administered with the anti-ulcer product. Natural biological products, e.g., Lactobacillus or Streptococcus faecium, and probiotics also are capable of restoring the natural balance of intestinal flora in affected young or adult animals. Suitable probiotics are commercially available as health supplements.

Other drugs, compounds, or substances may also be incorporated into the nano- or microbeads/particles and include, but are not limited to, mucosal protectants, such as sucralfate; a PGE₂ analog, e.g., misoprostol; vitamins, such as Vitamins A, B1, B2, B6, B12, D3, E and K, particularly Vitamin E; and/or plant extracts such as deglycyrrhizinated licorice, and Croton lechleri extract, or the aqueous soluble proanthocyanidin polymer or composition from Croton lechleri as described herein. In some embodiments, the compositions contain the C. lechleri proanthocyanidin polymer composition with an enteric coating.

In an embodiment, the microparticulate preparation has a core containing one or more anti-ulcer drugs, such as an antacids; proton pump inhibitors including, without limitation, omeprazole, rabeprazole, esomeprazole, or pantoprazole; and/or histamine H-2 receptor blockers, including, without limitation, ranitidine, famotidine, cimetidine and nizatidine. Also included in the core may be a C. lechleri proanthocyanidin polymer or C. lechleri extract. The drugs (and C. lechleri proanthocyanidin polymer or extract) may be in an immobilized form in the core, which is encapsulated within the polysaccharide-containing layer or coating, which is, in turn, covered or overlaid with a coating, such as an enteric coating, of the pH sensitive polymer. In embodiments, the enteric coating may be an alkali soluble, acid insoluble polymer, a high molecular weight polymer, or a combination of polymers, which prevent drug release prior to reaching the colon.

The one or more antacid drugs (and C. lechleri proanthocyanidin polymer or extract) may be immobilized in the core or the microparticulate preparation, for example and without limitation, within a swellable gel matrix, e.g. formed from gelatin, cellulose or derivatives thereof, alginate/alginic acid, k-carrageenan, or gel-forming synthetic polymers, such as polyamides or chitosan. The immobilization of the one or more drugs within a core can be achieved by admixing the drugs with compounds that can form a gel under certain conditions, wherein the drugs are then entrapped within the formed gel matrix. In general, immobilized forms of the antacid, proton pump inhibitor drugs as well as the C. lechleri proanthocyanidin polymer or extract refer to these active materials as being retained within a gel-like, viscous, or fluid material, enclosed or encapsulated within a coating, barrier, or outside layer, which may be porous or semi-permeable. This allows the active materials to pass out of the gel and be released from the microparticulate component. The immobilized forms may be adsorbed onto adsorbing agents or bound to chelating agents. Processes and techniques for immobilizing drugs or biologically active materials, which retain their biological activity after immobilization and/or release, are known and can be found, for example, in U.S. Pat. No. 5,356,625.

The multiparticulate component may comprise a preparation or composition that includes, illustratively and without limitation, powders; crystals; granules; small particles, including particles sized on the order of micrometers, e.g., microspheres and microcapsules; particles sized on the order of millimeters, or particles sized on the order of nanometers, e.g., nanoparticles; or microtablets, which are either hard or soft. The microparticles typically range in average size from about 25 μm to about 500 μm, or from about 50 μm to about 350 μm, or from about 300 μm to about 500 μm. Techniques for producing microparticles or microgranules containing releasable materials are known and are provided in the art, for example, in U.S. Pat. Nos. 5,356,625, 5,026,559 and 4,983,401. Such microparticulate components of the compositions described herein allow the antacid drug and C. lechleri proanthocyanidin polymer or C. lechleri extract materials to be available for action in regions of the gastrointestinal tract, such as the intestine and the colon, while being sequestered and protected from inactivation in the highly acid stomach environment. Accordingly, the outer, enteric and pH sensitive coating of the microparticles is dissolved or digested when the microparticulate components of the composition reach the intestine and/or colon of the animal; this allows the fluids of the intestine and colon to degrade the water soluble, carbohydrate components of the encapsulating layer, which exposes the core and permits release of the active materials to have their effect in the desired regions of the digestive tract.

The skilled practitioner will readily understand that parameters such as the thickness of the layers or coatings of the compositions of the invention, e.g., the encapsulating layer or coating comprising high molecular weight polymers such as carbohydrates, and the layer or coating comprising a pH sensitive polymer material, e.g., methacrylic acid polymer, as well as the size of the core, control the rate at which the anti-ulcer drug and C. lechleri proanthocyanidin polymer or C. lechleri extract materials are available at the target site, and the location within the digestive tract, e.g., the colon, where these active materials are available to exert their effects.

### Components of Pharmaceutically Acceptable Compositions and Formulations for Ulcer Treatment

Pharmaceutically acceptable preparations and formulations for ulcer treatment as described herein may further include any type of pharmaceutically acceptable excipients, additives, carriers, or vehicles. By way of nonlimiting example, diluents or fillers, such as dextrates, dicalcium phosphate, calcium sulfate, lactose, cellulose, kaolin, mannitol, sodium chloride, dry starch, sorbitol, sucrose, inositol, powdered sugar, bentonite, microcrystalline cellulose, or hydroxypropylmethylcellulose can be added to the composition (e.g., a C. lechleri proanthocyanidin polymer composition) to increase the bulk of the composition. In addition, binders, such as, but not limited to, starch, gelatin, sucrose, glucose, dextrose, molasses, lactose, acacia gum, sodium alginate, extract of Irish moss, panwar gum, ghatti gum, mucilage of isapgol husks, carboxymethylcellulose, methylcellulose, polyvinylpyrrolidone, Veegum and starch arabogalactan, polyethylene glycol, ethylcellulose, and waxes, can be added to the formulation to increase its cohesive qualities. Further, lubricants, such as, but not limited to, talc, magnesium stearate, calcium stearate, stearic acid, hydrogenated vegetable oils, polyethylene glycol, sodium benzoate, sodium acetate, sodium chloride, leucine, carbowax, sodium lauryl sulfate and magnesium lauryl sulfate can be added to the formulation. Also, glidants, such as, but not limited to, colloidal silicon dioxide or talc can be added to improve the flow characteristics of a powdered formulation. Disintegrants, such as, but not limited to, starches, clays, celluloses, algins, gums, crosslinked polymers (e.g., croscarmelose, crospovidone, and sodium starch glycolate), Veegum, methylcellulose, agar, bentonite, cellulose and wood products, natural sponge, cation-exchange resins, alginic acid, guar gum, citrus pulp, carboxymethylcellulose, or sodium lauryl sulfate with starch can also be added to facilitate disintegration of the formulation in the intestine.

In embodiments, the compositions of the invention are formulated with a substance that protects the antacid, proton-ion pump inhibitor and C. lechleri proanthocyanidin polymer and/or the polymer composition or C. lechleri extract from the stomach environment. For such protection, the components of the multiparticulate system and/or the proanthocyanidin polymer or extract composition may be enteric coated. Enteric coatings are those coatings that remain intact in the stomach, but will dissolve and release the contents of the dosage form once it reaches the small intestine. A large number of enteric coatings are prepared with ingredients that have acidic groups such that, at the very low pH present in the stomach, i.e., pH 1.5 to 2.5, the acidic groups are not ionized and the coating remains in an undissociated, insoluble form. At higher pH levels, such as in the environment of the intestine, the enteric coating is converted to an ionized form, which can be dissolved to release the proanthocyanidin polymer composition. Other enteric coatings remain intact until they are degraded by enzymes in the small intestine, and others break apart after a defined exposure to moisture, such that the coatings remain intact until after passage into the small intestines. A variety of polymers are useful for the preparation of enteric coatings, and the application of an enteric coating to the C. lechleri proanthocyanidin polymer composition, or extract, for example, can be accomplished by any method known in the art for applying enteric coatings, as may be found, for example, and without limitation, in U.S. Pat. Nos. 7,323,195 and 7,341,744, incorporated herein by reference. A C. lechleri proanthocyanidin polymer or extract composition in the form of granules and powder may be prepared using any method known in the art, such as, but not limited to, crystallization, spray-drying or any method of comminution, preferably using a high speed mixer/granulator, as described, for example and without limitation, in U.S. Pat. No. 7,323,195, incorporated herein by reference.

In other embodiments, the composition comprising antacid drugs and C. lechleri proanthocyanidin polymer or extract may be formulated as an aqueous suspension in admixture with suitable excipients. Non-limiting examples of excipients that are suitable for the manufacture of aqueous suspension include suspending agents, for example, methylcellulose, sodium carboxymethylcellulose, hydroxypropylmethycellulose, sodium alginate, polyvinyl-pyrrolidone, gum tragacanth and gum acacia; dispersing or wetting agents, which may be a naturally-occurring phosphatide, e.g., lecithin, or condensation products of an alkylene oxide with fatty acids, e.g., polyoxyethylene stearate, or condensation products of ethylene oxide with long chain aliphatic alcohols, e.g., heptadecaethyleneoxycetanol, or condensation products of ethylene oxide with partial esters derived from fatty acids and a hexitol, for example, polyoxyethylene sorbitol monooleate, or condensation products of ethylene oxide with partial esters derived from fatty acids and hexitol anhydrides, such as polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, e.g., sucrose, saccharin or aspartame. Dispersible powders and granules suitable for the preparation of an aqueous suspension by the addition of water can be used for components of the composition in admixture with a dispersing or wetting agent, suspending agent and one or more preservatives. Suitable dispersing or wetting agents and suspending agents are exemplified by those stated above. Additional excipients, for example, sweetening, flavoring and coloring agents, may also be present.

In an embodiment, the ulcer treatment composition including antacid and/or proton pump inhibitors and the C. lechleri proanthocyanidin polymer or extract is in the form of a gel, paste, or gel paste formulation, and associated microparticulate components as described herein. In an embodiment, the gel or paste is contained or preloaded in a delivery device, such as a syringe, e.g., a needle-less syringe, or other type of applicator or delivery system, especially for oral delivery. In an embodiment, the gel is not contained in a delivery device, but is administered to the roof of the mouth of the animal, particularly one that is too incapacitated or ill to eat or drink.

In an embodiment as described above, the gel or paste comprises pH-sensitive polymeric particles, such as microparticles or nanoparticles, to allow for pH-dependent uptake of the active compound into cells and/or the pH-dependent release of the active compound in different pH environments in an animal. Processes for generating granules and particles comprising the anti-ulcer drugs and the C. lechleri proanthocyanidin polymer or extract-containing composition, or a compressible form thereof, are as known and practiced in the art, and are as provided, for example, in U.S. Pat. No. 7,341,744, the contents of which are incorporated by reference herein. In an embodiment, gels or pastes are prepared for oral delivery and may contain copolymers, such as poloxamers and Pluronics of different types, e.g., Pluronic F.

In a particular embodiment, the anti-ulcer drugs and the C. lechleri proanthocyanidin polymer or extract, or a composition of proanthocyanidin polymer or extract, are present, in combination, in a gel or paste composition or formulation, preferably for oral administration. Other ingredients, such as cyto- or mucoprotective drugs or agents may be included in the composition or formulation. By way of example, the gel or paste may comprise, without limitation, an oily vehicle or excipient, such as a hydrophobic oily vehicle, a basifying agent, a flavoring agent and a coloring agent. Such a gel or paste is particularly suitable for oral administration. Illustrative and nonlimiting examples of hydrophobic oily vehicles include vegetable oil, triglyceride or polypropylene glycol, as well as a thickening agent, e.g., aluminum stearate. Flavoring agents can include, for example, fruit flavors, mint flavors, honey flavor, and other natural and organic flavorings known to those skilled in the art. Coloring agents can include, for example, iron oxide or titanium dioxide. Alternatively, the oily vehicle can be liquid paraffin or other suitable waxes, including a thickening agent.

Oily suspensions may be formulated by suspending the anti-ulcer drugs and C. lechleri proanthocyanidin polymer as active ingredients in a vegetable oil, e.g., arachis oil, olive oil, sesame oil or coconut oil, or in mineral oil, such as liquid paraffin. The oily suspensions may contain a thickening agent, e.g., beeswax, hard paraffin or cetyl alcohol. Oral preparations can include sweetening agents as mentioned above and flavoring agents to improve palatability. Pharmaceutically acceptable preservatives, for example, an anti-oxidant such as ascorbic acid, can also be added to such compositions.

The anti-ulcer drug- and C. lechleri proanthocyanidin polymer or extract-containing pharmaceutical compositions used in the methods of the invention may also be in the form of an oil-in-water emulsions. The oily phase may be a vegetable oil such as olive oil or arachis oil, or a mineral oil such as liquid paraffin or mixtures of these oils. Examples of emulsifying agents include, without limitation, naturally-occurring phosphatides, e.g., soy bean, lecithin, and esters or partial esters derived from fatty acids and hexitol anhydrides, e.g., sorbitan monooleate, and condensation products of partial esters with ethylene oxide, e.g., polyoxyethylene sorbitan monooleate. Sweetening, coloring and flavoring agents can be included in the emulsions.

Syrups and elixirs containing the anti-ulcer drugs in combination with the C. lechleri proanthocyanidin polymer or C. lechleri extract may also can be formulated with sweetening agents, for example, glycerol, propylene glycol, sorbitol or sucrose. Such formulations may also contain a demulcent, a preservative and flavoring and coloring agents. The pharmaceutical compositions may be in the form of a sterile, orally deliverable or administrable aqueous or oleagenous suspension. This suspension may be formulated according to methods known in the art using suitable dispersing or wetting agents and suspending agents, such as those mentioned above. The sterile pharmaceutical preparation may also be a sterile solution or suspension in a non-toxic parenterally-acceptable diluent or solvent, for example, a solution in 1,3-butane diol. Illustrative, acceptable vehicles and solvents that may be used in the preparations include water, Ringer's solution and isotonic sodium chloride solution. Co-solvents, e.g., ethanol, propylene glycol or polyethylene glycols, may also be included. In addition, sterile, fixed oils, e.g., any bland, fixed oil such as synthetic mono- or diglycerides, are conventionally employed as solvents or suspending media and may be used. In addition, fatty acids, such as oleic acid and the like, may be used in injectable preparations.

### Methods of Treatment and Applications of Use

The invention is directed to methods of treating and preventing ulcers of the gastrointestinal region and especially the colon in young and adult animals, particularly equine animals like horses that can be naturally high-strung and can become stressed as a result of events in their habitats and lifestyles, as well as from endurance activities and performances expected of them. The methods also advantageously encompass the treatment of diarrhea associated with ulceratic conditions in afflicted animals. The method of the invention comprises administering to an animal in need of ulcer treatment or prevention, a pharmaceutically acceptable composition comprising one or more acid reducing or blocking drugs and a proanthocyanidin polymer or extract from a Croton species in amounts effective to treat the ulcers and/or the symptoms thereof. Advantageously, such compositions also treat diarrheal conditions that may concurrently affect the animal. In preferred embodiments, the proanthocyanidin polymer or extract is from a Croton species, namely, Croton lechleri. Treating the ulcers can involve reducing the severity and duration of the ulcers in the animal. Treating the ulcers can also involve decreasing the discomfort and pain associated with ulceratic lesions in the animal undergoing treatment. Treating the ulceratic animals with compositions comprising the proanthocyanidin polymer or extract from Croton lechleri also results in treating a diarrheal condition that may result from or accompany the ulceratic condition, as well as in improving the overall basic and gastrointestinal health of the animals, which may ultimately reduce their morbidity and mortality.

In an embodiment of the invention, the methods are directed to treating or preventing ulcers in non-human young and adult animals suffering from ulcers or at risk of developing ulcers due to environmental conditions or lifestyle activities. The methods of the invention further relate to the treatment or prevention of ulcers and/or related symptoms in adult, non-human animals, such as, without limitation, adult equine animals, exotic animals, and domestically or commercially used animals. Often, but not always, the animals are large in size and have complex gastrointestinal systems. For example, in horses, which are optimally suited for the treatment methods of the invention, the intestinal volume of the animal is large, with the main sites of ulceratic lesions often being in the stomach, duodenum and colon. Consequently, the described methods provide treatment of ulcers with anti-ulcer, acid blocking or reducing agents or drugs in combination with a proanthocyanidin polymer or extract from C. lechleri, or composition thereof, providing for an adequate amount and appropriate distribution of the anti-ulcer agents or drugs, e.g., antacids and proton pump inhibitors, and proanthocyanidin polymer in the gut of the animal so as to treat or prevent the ulcers and/or their symptoms, and optimally cure the ulcers in the animal.

In a related manner, ruminant animals, such as camels, which may also be treated by the methods of the invention as needed, possess multi-chambered stomachs, e.g. four stomach compartments, including a rumen or first compartment of the alimentary canal, which serves as the primary site for microbial fermentation of ingested feed. The described methods provide treatment of ruminant animals such as camels, sheep and goats with the anti-ulcer compositions including antacids and proton pump inhibitors in combination with a proanthocyanidin polymer from C. lechleri, providing for an adequate amount and appropriate distribution of the acid reducing or blocking agents and the proanthocyanidin polymer in the intestine/hindgut of the animal, rather than the rumen, so as to treat the ulcers in particular regions of the animal's digestive system and/or the related symptoms and optimally cure the animal.

The non-human young and adult animals for which the treatment methods are suitable may include different animal types, genera, or species. In general, young and adult farm animals, animals bred or kept for various purposes, such as sport (e.g., racing, riding, dressage), transport, domestic, companion, industrial uses (e.g. hauling, pulling, plowing), and the like, are particularly amenable to treatment according to the methods of the invention. For example, encompassed by the methods of the invention is the treatment of adult or young non-human animals, such as camels (calves), sheep (lambs), rams, horses (foals), pigs (piglets), goats (kids), bison/buffalo (calves), llamas, donkeys, mules, yaks, etc. Neonatal, young and adult exotic animals, such as zoo animals of various species, are also embraced by the treatments of the invention. In preferred aspects, young and adult horses are animal subjects that are particularly amenable to the methods and compositions of the invention.

### Dosage Forms and Administration

In certain embodiments, the proanthocyanidin polymer composition is provided in the ulcer treatment compositions in an amount of 50 to 600 mg, in an amount of 200 to 300 mg, or in an amount of 250 mg. Those skilled in the art will appreciate that due to the higher purity of compositions such as SP-303 or crofelemer and SB-300, more by weight of SB-300 than SP-303 will need to be used in compositions to achieve the same amount of the active ingredient of the proanthocyanidin polymer composition. SB-300 generally has about 67% by weight of the proanthocyanidin polymer composition while SP-303 has higher purity, for example 99-100%. By way of example, antacids, e.g., ranitidine, and proton pump inhibitors, e.g., omeprazole, may be present in the compositions in an amount of from 20 mg to 1500 mg; or from 50 mg to 1000 mg, from 100 mg to 500 mg, or from 10 mg to 250 mg, and amounts in between. Illustratively, ranitidine is typically administered in an amount of 6.6 mg/kg TID (three times per day), and omeprazole is typically administered in an amount of from 1 to 4 mg/kg SID (once per day). Based on commonly-used normal and typical doses of antacids and proton pump inhibitors, it is within the purview of one having skill in the art to determine the appropriate amount of these components for providing and administering to an animal in need in the compositions and methods of the invention.

The routes of administration of the anti-ulcer drug- and C. lechleri proanthocyanidin polymer-containing compositions to afflicted animals are not intended to be limiting. However, routes of administration amenable to optimally dispensing or administering the paste, gel, or gel paste compositions with associated microparticulates to the gastrointestinal and cecum-colonic regions of the animal are preferred. By way of example, oral or buccal administration, including, without limitation, roof of mouth, dental, periodontal, or esophageal administration are encompassed by the invention. In embodiments, food source (animal feed), nutrition source, libation source, or food and/or drink supplement could be used. In an embodiment, the combination product could be provided in an aqueous formulation, administered to the animal as a drench or directly from a ready-to-use (RTU) bottle directed to the esophageal cavity so as to more effectively reach the animal's intestine/gut for optimal activity. In a related embodiment, administration can also be by inclusion in the regular or special diet of the animal, such as in a functional food for the animals in need, or as a dietary supplement or food supplement, e.g., as described in WO 00/47062, for administration to an animal in need thereof according to the present invention. In other embodiments, the compositions of the invention can be formulated for rectal administration, such as a suppository, enema or other convenient form. The compositions can also be provided as a controlled release system (See, e.g., Langer, 1990, Science 249: 1527-1533).

In other embodiments, for oral administration, the anti-ulcer drug- and C. lechleri proanthocyanidin polymer-containing product may be encapsulated and formulated with suitable carriers, and the like, in solid dosage forms. Nonlimiting examples of suitable carriers, excipients, diluents and vehicles include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, edible oils, and the like. The formulations can also include lubricating agents, wetting agents, emulsifying and suspending agents, preserving agents, sweetening agents or flavoring agents. As indicated, the compositions can be formulated to provide rapid, sustained, extended, or delayed release of the active ingredients after administration to the animal by employing protocols and methods well known in the art. The formulations can also include compounds or substances that reduce proteolytic degradation and promote absorption such as, for example, surface active agents.

As will be appreciated by those having skill in the art, the specific dose can be calculated according to the approximate body weight, body mass, or body surface area of the animal, or the volume of body space or mass to be occupied. The dose also depends on the particular route of administration selected by the practitioner. Further refinement of the calculations necessary to determine an appropriate dosage for treatment is routinely made by those of ordinary skill in the art, for example, using appropriate assays and analytical procedures, such as has been described for certain compounds (e.g., Howitz et al., 2003, Nature, 425:191-196). Exact dosages can be determined based on standard dose-response studies. Therapeutically effective doses for treatment of afflicted animals can be determined, by titrating the amount of the active product given to the animal to arrive at the desired therapeutic effect, while minimizing side effects.

For use in treating ulcers and associated conditions such as diarrhea in young animals, e.g., foals, in accordance with the methods of the invention, a therapeutically acceptable form of the C. lechleri proanthocyanidin polymer or extract in the compositions is administered, particularly orally administered, in an amount ranging from 0.1 to 100 mg/kg per day, once, twice or more daily. In other embodiments, the amount can range from about 0.1 to about 10 mg/kg/day, once, twice or more daily; or from about 0.1 to about 25 mg/kg/day, once, twice or more daily; or from about 0.1 to about 30 mg/kg/day, once, twice or more daily; or from about 0.1 to about 40 mg/kg/day, once, twice or more daily. In other embodiments, the dose can be 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, etc., as well as incremental dose amounts in between. In still other embodiments, the amount can range from about 1 to about 10 mg/kg/day once, twice or more daily; or from about 1 to about 5 mg/kg/day, from about 1 to about 8 mg/kg/day, from about 1 to about 10 mg/kg/day, or from about 2 to about 4 mg/kg/day once, twice or more daily. In other embodiments, the foregoing amounts of the C. lechleri proanthocyanidin polymer composition or extract are administered, for example, twice daily, three times daily, four times daily, or more than four times daily, rather than once per day. Higher doses, e.g., 50 mg/kg or 100 mg/kg per day or twice or more daily, may be required, as necessary, to treat diarrhea and accompanying dehydration in young animals.

In other embodiments, for the treatment methods, a suitable dose for the C. lechleri extract, in particular, the C. lechleri proanthocyanidin polymer product, or the C. lechleri proanthocyanidin polymer composition, such as SP 303 or SB 300, in combination with one or more anti-ulcer drugs in the compositions described herein may range from about 1 mg to about 1000 mg, or from about 10 mg to about 500 mg, or from about 50 mg to about 350 mg, or from about 30 mg to about 400 mg, or from about 100 mg to about 250 mg, or from about 50 mg to about 300 mg. It will be understood that the ranges include the lower and higher amounts specified, as well as amounts in between. The compositions can be administered once a day, or multiple times per day, as appropriate or required. Doses administered once or multiple times per day can be given for consecutive days, e.g., two days, three days, four days, five days, six, days, seven days, or more, in some embodiments. A dose administered multiple times per day may embrace two, three, four, five, six, ten, or more times per day. Other dosing schedules, such as every other day, or every third day, every fourth day, etc. are embraced by the invention. In addition, one having skill in the art will appreciate that doses and amounts administered to the animal can vary, given the wide range of weights of the animals undergoing treatment, as well as the animal species and type of digestive system, e.g., ruminant or non-ruminant. In an embodiment the C. lechleri proanthocyanidin polymer co-formulated with anti-ulcer drugs in the composition is SB 300, either enterically or non-enterically coated for treatment of ulcers in horses.

In some embodiments, daily doses, including multiple daily doses, e.g., twice or three times a day, of the C. lechleri proanthocyanidin polymer product or extract, and amounts of the proanthocyanidin in the compositions of the invention may be 2 mg, 5 mg, 10 mg, 15 mg, 20 mg, 25 mg, 50 mg., 100 mg, 150 mg, 175 mg, 200 mg, 225 mg, 250 mg, 275 mg, 300 mg, 325 mg, 350 mg, 375 mg, 400 mg, 500 mg (or amounts there between) per animal. Administration schedules may also be altered to achieve a therapeutically effective concentration of the C. lechleri proanthocyanidin polymer or C. lechleri extract to treat ulcers and diarrhea and their symptoms as described herein. By way of specific, yet nonlimiting, example, a suitable dosage amount of C. lechleri proanthocyanidin polymer for use in the methods according to the invention is 250 mg once or twice daily. Daily doses of anti-ulcer drugs and amounts for use in the compositions, can be ascertained by the skilled practitioner. By way of nonlimiting example, daily doses of antacids, e.g., H-2 receptor blockers and proton pump inhibitors, e.g., omeprazole, and amounts of these actives in the compositions of the invention, may be 2 mg, 5 mg, 10 mg, 15 mg, 20 mg, 25 mg, 50 mg., 100 mg, 150 mg, 175 mg, 200 mg, 225 mg, 250 mg, 275 mg, 300 mg, 325 mg, 350 mg, 375 mg, 400 mg, 450 mg, 500 mg, 550 mg, 600 mg, 700 mg, 1000 mg, 1500 mg (or amounts there between) per animal.

It will be understood that the dose amount actually administered can be determined by the practitioner, in the light of the relevant circumstances, including the severity of the ulceratic condition, and/or symptoms thereof being treated, the age, weight, and response of the individual animal receiving treatment, as well as the chosen route of administration.

### EXAMPLES

### Example 1

### Determining Foregut Versus Hindgut Differential Diagnosis of Gastrointestinal Conditions in the Horse Related to the Treatment of Gastric or Colonic Ulcers, or Both

Because variations in disease, disease etiology and function exist between the equine foregut and hindgut, treatment that is localized to the gastrointestinal region affected by an ulceratic condition, or a potential ulceratic condition, is important in order to be most effective. A first step in evaluating the health of the equine digestive tract should is determining if an ulcerative condition exists and where it exists—in the foregut, hindgut, or both. Particular pathological conditions may be associated more with the animal's foregut than the hindgut, and vice versa. Moreover, external symptoms such as lack of appetite, poor condition, and performance issues, may be the same for conditions affecting the foregut and the hindgut. However, while the signs can be similar from foregut to hindgut and among pathologies, the treatment approaches may vary widely.

Among the conditions that afflict the equine foregut, gastric ulcers may be the most widely suspected and diagnosed given the high incidences of gastric ulcers and widespread reporting of such ulcers. Stomach ulcers are generally understood to represent a syndrome (Equine Gastric Ulcer Syndrome, or EGUS) and may be typically associated with other, underlying disease states, which may include gastritis, neoplasia, parasitism, anterior enteritis, and others. Among the possible conditions that afflict the equine hindgut, colonic ulcers have been reported as a relatively widespread condition in recent years. Just as lesions of the stomach reflect other underlying conditions, colonic ulcers may also be associated with colitis, intestinal traumas (rectal tears, traumatic events), parasitism, neoplasia, and intestinal inflammatory disease.

While the above-noted external symptoms constitute initial signs that some type of gastrointestinal health problem may be impacting the behavior and performance of a horse, such indicators are vexingly non-differential. As a result, these symptoms may be assumed to reflect gastric conditions when, in fact, they are as likely to be associated with hindgut issues in the animal. Also, a horse that presents with these symptoms may be suffering from a foregut issue, a hindgut issue, or both. Therefore, symptoms alone are not reliable in supporting an accurate differential diagnosis.

The guaiac acid fecal occult blood test (gFOBT) was found to have good sensitivity and specificity when results were confirmed at post mortem. Guaiac acid works by binding hemoglobin which induces a detectable color change. It was found to be 65% accurate for diagnosing the presence of either gastric or colonic ulcers. However, false negatives are common ((F. L. Pellegrini et al., 2005, J. Equine Veterinary Science, 25(3):133-117). Another type of test, the non-invasive fecal blood test (FBT), is a rapid antibody-based test system that is able to detect equine albumin and hemoglobin in a fecal sample. A commercial FBT product for horses is known as SUCCEED®, which is reported to distinguish foregut and hindgut lesions. The SUCCEED® FBT test is comprised of two assays. Test A in the FBT assay employs antibodies directed against equine albumin and can indicate bleeding in the hindgut; Test H in the assay employs antibodies directed against equine hemoglobin; a positive result for the hemoglobin detection test is said to be indicative of bleeding from anywhere in the GI tract.

In an assay such as the FBT, albumin serves as a proxy for hindgut lesions. While ulceratic lesions in the mucosal lining anywhere along the GI tract can produce albumin, albumin produced in the stomach is digested by acids and enzymes in the proximal portion of the small intestine. Thus, the presence of albumin in feces reflects a source that is caudal to the common bile duct or, generally, the hindgut. Hemoglobin can be present with any injury severe enough to produce whole blood. Because it is resistant to digestive acids and enzymes, the presence of hemoglobin in feces can reflect injury anywhere in the GI tract. Used together, the two parts of the SUCCEED® FBT test differentiate between foregut and hindgut lesions.

Whether or not a horse is symptomatic, use of the gFBOT or FBT to identify whether lesions are present in the foregut, hindgut, or both, at the beginning of the diagnostic process assists in tailoring the treatment to allow the targeting of ulcers that are present in distinct parts of the gastrointestinal tract. Such fecal blood testing as part of equine wellness examinations may provide an early warning signal and an initial indicator that points to gastrointestinal ulceration, or potential gastrointestinal ulceration, in the foregut, hindgut, or both, of the animal. This type of initial assay can then guide further diagnostics, e.g., gastroscopy, CBC, fecal egg counts, ultrasound, etc. to achieve more accurate or definitive diagnoses and, thus, to utilize the methods and compositions of the present invention as targeted treatment for both gastric and colonic ulcers, and particularly, for colonic ulcers in horses.

### Example 2

### Evaluation of the Effect of Administration of an Anti-Ulcer Treatment on Ulcers and Ulceratic Conditions in Horses Undergoing Intensive Training

A study is conducted to evaluate the effect of an anti-ulcer treatment product in the treatment of gastric, duodenal and/or colonic ulcers and related conditions in horses in intensive training, and/or in horses known to have gastric ulcers.

The study design involves a masked, randomized, negative controlled, single site field study in which horses known to possess gastric ulcers are treated. Participating horses are randomly allocated to different treatment groups, including a control group. Ther horses are dosed for 28 consecutive days and undergo gastroscopy every 14 days, during which gastric ulcer scores and gastric pH are recorded. Gastroscopy with a 3-meter endoscope is performed as described by White et al. (2007, J Am. Vet. Med. Assoc., 230:1680-1682) to detect and score gastric ulcers. Gastric ulcers may be classified into the following grades: Grade 0: normal, unulcerated tissue. The epithelium is intact, and there is no thickening or abnormal coloring. Grade 1: The mucus lining is intact, but there are areas of thickened, discolored tissue. Grade 2: Small, single or multiple ulcers are present. Grade 3: Large, single or multiple ulcers are present. Grade 4: Extensive, deep ulcers are present. (F. L. Pellegrini, 2005, J. Equine Vet. Sci., 25(3):113-117). Improvement of squamous ulcers is defined by a reduction in the Gastric Ulcer Council Scores by 1 grade or more (e.g. going from Grade 3 to Grade 2). Improvement of glandular ulcers is defined as a decrease in severity (e.g., a change from moderate to mild) and improvement in description (e.g., a change from raised and hemorrhagic to flat and hemorrhagic).

A fecal occult blood test (FOBT), in particular, a guaiac-based fecal occult blood test (gFOBT), is used in all animals. In the absence of gastric ulcers, a positive gFOBT has a positive predictive value of 100% for colonic ulcers (F. L. Pellegrini, 2005, J. Equine Vet. Sci., 25(3):113-117). Abdominal ultrasonography of the right dorsal colon (Jones et al., 2003. J. Am. Vet. Med. Assoc., 222:1248-1251) is performed in all horses to determine the incidence of mural thickening that is indicative of colonic ulcers (normal=<4 mm). Clinical scoring for general health and fecal dry matter content measurement is also used to complete the evaluation.

As described herein, the anti-ulcer product is a paste or a gel formulation. In particular formulation, the paste or gel formulation has at least two phases or components that are associated. The first phase or component is a liquid or oil based paste or gel which contains antacids and/or acid blockers such as proton pump inhibitors, examples of which include omeprazole, rabeprazole, esomeprazole, or pantoprazole, and/or histamine H-2 receptor blockers, examples of which include ranitidine, famotidine, cimetidine and nizatidine. Antacids which may be included are typically those used in the art, such as salts or calcium, aluminum, magnesium and sodium and mixtures thereof, as described herein. Cytoprotective agents, such as sucralfate, misoprostol and bismuth subsalicylate and/or natural products, such as deglycyrrhizinated licorice and vitamins, such as Vitamins A, B1, B2, B6, B12, D3, E and K, particularly Vitamin E; and/or extract of Croton lechleri, such as the aqueous soluble proanthocyanidin polymer from Croton lechleri as described herein, can also be included.

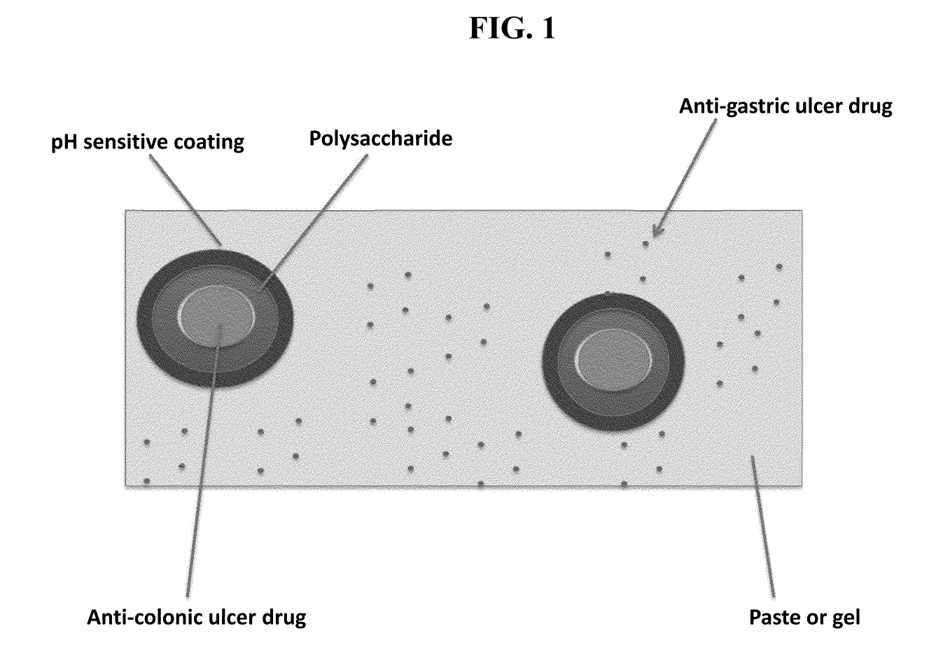

The second phase or component associated with the gel or paste includes a colon-targeted delivery system comprised of a multiparticulate system, such as microspheres, microparticles, and/or nanoparticles containing anti-ulcer drugs and other incorporated components. The drugs and other components form a core portion of the microparticulate system, which has two protective layers or coatings: a pH-sensitive polymer, such as EUDRAGIT® methacrylate polymers, to prevent the burst release of drugs in the stomach or the proximal part of the small intestine (the duodenum) and a high molecular weight carbohydrate, e.g., polysaccharide, layer or coating to allow drug release in different parts of the cecum-colon after hydrolysis by bacterial enzymatic degradation. A representation of the composition for treating ulcers in animals in need is shown in FIG. 1. In the compositions of the invention, it will be appreciated that the microparticulate system, particularly the core, may contain one or more antacid drugs, including H-2 receptor antagonists, proton pump inhibitors, and aqueous soluble proanthocyanidin polymer from Croton lechleri as actives in treating ulcers of the stomach, intestine and colon, as well as diarrhea, in the animals in need.

If the drug itself does not readily bind to the surface of the ulcer in the colon, for example, by attaching to an exposed protein like sucralfate, an additional phase or layer comprising liposomes or mucoadhesive drug carriers, such as chitosan-based or coated microparticles, may be added to the microparticulate component to increase local drug delivery to inflamed tissues.

The pH sensitive polymer layer or coating may include, without limitation, pH sensitive polymers such as methacrylic acid polymers (EUDRAGIT®), cellulose acetate phtalates, pH controlled silicone microspheres, sodium alginate, or pH responsive gelatins. To avoid the premature release of the drug in the duodenum or ileum, which might result in drug loss before reaching the colon, release combined polymers, for example, EUDRAGIT® and poly(D,L-lactide-co-glycolide), or newer-generation polymers, for example, EUDRAGIT® P4135-F that can dissolve at pH greater than 7.2, may be used.

The polysaccharide can include, for example, cellulose for release in the cecum by cellulolytic bacteria and/or starch for release in other parts of the colon. Other polymers may be from plants, such as ethyl cellulose, pectin, inulin, locust bean gum, guar gum, amylose; from algae, such as alginates; from fungi, such as sleroglucan; from bacteria, such as dextran, cyclodextrin; or from animals, such as chondroitin sulfate, hyaluronic acid, or chitosan. To optimize the release profile and adapt to the specific conditions of the horse hind gut, a mixed coating or layer may be used, such as, for example, a coating or layer comprised of pectin-ethylcellulose, guar gum-ethylcellulose, amylose-ethylcellulose, pectin-chitosan, polysaccharide-EUDRAGIT®, or pectin-chitosan-hydroxyproplymethylcellulose (HPMC).

The composition of the colonic microflora can also be altered in disease conditions such as colitis and colonic ulcers. Accordingly, the composition of the coating will be specific to the disease condition. To increase the efficiency of the bacterial enzyme degradation, a probiotic which contains bacteria producing one or more enzymes suitable or appropriate for a given polysaccharide may be co-administered with the anti-ulcer product.

Other drugs, compounds, or substances to be incorporated into the nano- or microbeads/particles may include, but are not limited to, mucosal protectants, such as sucralfate; a PGE₂ analog, e.g., misoprostol; vitamins, e.g., Vitamins A, B1, B2, B6, B12, D3, E and K, particularly Vitamin E; a plant extract such as deglycyrrhizinated licorice; or aqueous soluble C. lechleri proanthocyanidin polymer or extracts of Croton lechleri, as described herein.

All patents, patent applications and publications referred to or cited herein are hereby incorporated by reference in their entireties for all purposes.

It is understood that the embodiments and examples described herein are for illustrative purposes and that various modifications or changes in light thereof will be suggested to persons skilled in the pertinent art and are to be included within the spirit and purview of this application and scope of the appended claims. It is to be understood that suitable methods and materials are described herein for the practice of the embodiments; however, methods and materials that are similar or equivalent to those described herein can be used in the practice or testing of the invention and described embodiments.

1. A method of treating ulcers and/or symptoms related thereto in a large, non-human animal, the method comprising: administering to the non-human animal in need thereof a pharmaceutically acceptable composition comprising one or more agents that reduce or inhibit acid production in the gastrointestinal tract of the animal, in combination with an aqueous soluble proanthocyanidin polymer from Croton lechleri, a Croton lechleri extract, or a pharmaceutically acceptable composition thereof, in effective amounts to treat the ulcers and/or the related symptoms in the animal.

2. The method according to claim 1, wherein the animal has, or is at risk of developing, ulcers or ulceratic conditions and/or the symptoms thereof.

3. The method according to claim 1, wherein the C. lechleri proanthocyanidin polymer is an enteric coated pharmaceutical composition.

4. The method according to claim 1, wherein the C. lechleri proanthocyanidin polymer is a non-enteric pharmaceutical composition.

5. The method according to claim 1, wherein the one or more agents that reduce or inhibit acid production is a proton pump inhibitor.

6. The method according to claim 5, wherein the proton pump inhibitor is selected from omeprazole (PRILOSEC, GASTROGARD, ULCERGARD), rabeprazole (ACIPHEX, ACIFIX), esomeprazole (NEXIUM), or pantoprazole (PROTONIX).

7. The method according to claim 6, wherein the proton pump inhibitor is omeprazole (PRILOSEC, GASTROGARD, ULCERGARD).

8. The method according to claim 1, wherein the one or more agents that reduce or inhibit acid production is an antacid.

9. The method according to claim 8, wherein the antacid is selected from magnesium, aluminum, sodium and calcium salts and mixtures thereof.

10. The method according to claim 1, wherein the one or more agents that reduce or inhibit acid production is an H-2 receptor antagonist.

11. The method according to claim 10, wherein the H-2 receptor antagonist is selected from cimetidine (TAGAMET), ranitidine (ZANTAC), nizatidine (AXID, AXID AR), or famotidine (PEPCID).

12. The method according to claim 1, wherein the composition containing the one or more acid reducing or inhibiting agents and the C. lechleri proanthocyanidin polymer or C. lechleri extract is orally administered to the non-human animal.

13. The method according to claim 1, wherein ulcers and diarrhea are treated in the animal.

14. The method according to claim 1, wherein the non-human animal is a juvenile animal.

15. The method according to claim 1, wherein the non-human animal is an adult animal.

16. The method according to claim 14, wherein the non-human animal is an equine animal.

17. The method according to claim 16, wherein the equine animal is a foal or an adult horse.

18. (canceled)

19. The method according to claim 13, wherein the animal is afflicted with one or all of gastric ulcers, intestinal ulcers, colonic ulcers and/or symptoms thereof.

20. The method according to claim 19, wherein the composition is formulated in the form of a gel, paste, or gel paste.

21. The method according to claim 20, wherein the gel, paste, or gel paste is administered to the animal by topical application to the roof of the animal's mouth.

22. The method according to claim 20, wherein the gel, paste, or gel paste is contained in a delivery device.

23. The method according to claim 22, wherein the delivery device is a syringe.

24. The method according to claim 20, wherein the gel, paste, or gel paste comprises polymeric microparticles or nanoparticles containing the one or more acid reducing or blocking agents.

25. The method according to claim 24, wherein the gel, paste, or gel paste comprises polymeric microparticles or nanoparticles containing the C. lechleri proanthocyanidin polymer or C. lechleri extract.

26. The method according to claim 25, wherein the polymeric microparticles or nanoparticles are pH-sensitive.

27. The method according to claim 25, wherein the C. lechleri proanthocyanidin polymer or extract is administered to the animal in an amount of at least 50 mg to 250 mg.

28. The method according to claim 1, wherein the symptoms associated with the ulcers or ulceratic condition in the animal include one or more of weight loss, decline in body condition; resistance under saddle; irritability; changes in attitude; lack of energy and stamina; loss of appetite; pawing indicative of discomfort; laying down excessively; diarrhea and low-grade anemia.

29. The method according to claim 27, wherein the C. lechleri proanthocyanidin polymer composition is selected from the group consisting of SB 300, SP 303 and crofelemer.

30. The method according to claim 29, wherein the C. lechleri proanthocyanidin polymer composition is SB 300.

31. The method according to claim 1, wherein the pharmaceutically acceptable composition comprises one or more agents that reduce or inhibit acid production in the gastrointestinal tract of the animal, in combination with an extract of C. lechleri.

32. The method according to claim 20, wherein the gel or paste is liquid or oil based.

33. The method according to claim 1, wherein the composition is chronically administered.

34. A multicomponent composition for simultaneously treating or preventing gastric, intestinal and colonic ulcers in a non-human animal in need, comprising:
a paste or gel component comprising one or more drugs that reduce, block, or inhibit acid production and/or increase pH of the gastrointestinal tract in combination with an aqueous soluble proanthocyanidin polymer from Croton lechleri or a Croton lechleri extract;
a microparticulate component admixed or associated with the paste or gel component, said microparticle component comprising:
one or more drugs that block or inhibit acid production and/or increase pH in the colon,
a layer or coating comprising one or more aqueous soluble, high molecular weight polymers; and
a layer or coating comprising a pH sensitive substance or agent;
wherein, in the microparticulate component, the high molecular weight polymer layer or coating encapsulates the one or more drugs that reduce or block acid production in the colon, thereby forming a core surrounded by an encapsulating layer; and wherein the layer or coating comprising the pH sensitive substance or agent surrounds the encapsulating layer, thereby forming an enteric outer coating; and
wherein the microparticulate component is in association with the paste or gel component so as to deliver the drugs to treat or prevent gastric, intestinal and colonic ulcers in the animal.

35. The composition according to claim 34, wherein the core further comprises one or more of cytoprotectants, mucosal protectants, prostaglandin E₂ (PGE₂) analog, vitamins, plant extracts, soluble proanthocyanidin polymer from Croton lechleri, or Croton lechleri extract.

36.-57. (canceled)

58. A method of treating or preventing at least one, or all, of gastric, intestinal and colonic ulcers in a non-human animal that has, or is at risk of developing, ulcers of the gastrointestinal tract, comprising administering to the animal in need thereof an effective amount of the composition according to claim 34.

59. The method according to claim 58, wherein the composition is orally administered.

60. The method according to claim 58, wherein the composition is administered to a young or adult equine animal that has, or is at risk of developing, one or more of a gastric, intestinal, or colonic ulcer and/or symptoms related thereto.

61. The method according to claim 60, wherein the animal is a horse.

62. The method according to claim 60, wherein the composition is administered once a day or multiple times a day for greater than one day.

63. The method according to claim 60, wherein the animal is further treated for diarrhea.

64. The method according to claim 58, wherein the composition is co-administered with a probiotic.

65. The method according to claim 64, wherein the probiotic contains microorganisms that produce specific enzymes which degrade the polysaccharide layer of the microparticulate component of the composition.

66. The method according to claim 58, wherein colonic ulcers are treated or prevented in the animal.
